(19) United States
(12) Patent Application Publication
(10) Pub. No.: US 2024/0209397 A1
(43) Pub. Date: Jun. 27, 2024

(54) Thermally Stable Lipid-Nucleic Acid Molecule Formulations Utilising Metal Organic Framework (MOF) Shells

(71) Applicant: Commonwealth Scientific and Industrial Research Organisation, Acton, Australian Capital Territory (AU)

(72) Inventors: Ruhani Singh, Clayton, Victoria (AU); Daniel Layton, Clayton, Victoria (AU); Cara Doherty, Clayton, Victoria (AU); Xavier Mulet, Clayton, Victoria (AU); Benjamin Muir, Clayton, Victoria (AU)

(21) Appl. No.: 18/287,604

(22) PCT Filed: May 17, 2022
(86) PCT No.: PCT/AU2022/050476

§ 371 Date: Oct. 19, 2023

(30) Foreign Application Priority Data
May 17, 2021 (AU) 2021901467

(51) Int. Cl. C12N 15/88; A61K 47/02; A61K 47/24; A61K 47/54; A61K 47/69; A61K 48/00

(52) U.S. Cl. CPC C12N 15/88; A61K 47/02; A61K 47/24; A61K 47/543; A61K 47/6911; A61K 48/0041

(57) ABSTRACT

The present application relates to metal-organic framework (MOF) encapsulation of lipid-nucleic acid formulations. The present application discloses methods for stabilizing lipid-nucleic acid formulations and provides MOF encapsulated lipid-nucleic acid formulations with improved stability.

### FIELD OF INVENTION

The present application relates to formulations and methods for stabilizing lipid-nucleic acid molecule formulations and use thereof for delivery of the molecule into cells.

### BACKGROUND

Intracellular delivery of nucleic acid molecules into cells is critical for several progressive therapeutic, clinical and research applications. Intracellular delivery of nucleic acid molecules can be achieved using multiple approaches, including viral (for example, adenoviruses, retroviruses, adeno-associated viruses, herpes simplex viruses and vaccinia viruses), chemical (for example, lipofection, calcium-phosphate, DEA-dextran) and physical (for example, electroporation, bombardment, microinjection) methods.

Lipid based delivery systems such as liposomes for example are able to facilitate intracellular nucleic acid delivery. A number of commercial and non-commercial carriers have been developed by combining different cationic lipids with a variety of helper lipids (e.g., DOPE or cholesterol) that can be mixed with nucleic acid to form a positively charged complex (lipoplex) for the delivery of negatively charged molecules. Lipoplexes are often unstable in solution, resulting in the formation of aggregates, which contribute to lower transfection efficiency. This is different to the analogous proteoliposome system, where a transmembrane protein is bound within a lipid bilayer, which results in inherent stability of the two molecules. This is due to the presence of a hydrophobic transmembrane region within the protein that associates directly with the lipid bilayer. Conversely, the complexing of a nucleic acid to a lipid in a lipoplex requires the formation of a weak bond (e.g. electrostatic) which is easily broken and results in low stability and is particularly sensitive at ambient temperatures.

There is therefore a need for increasing the storage stability of lipid-nucleic acid molecule products.

### SUMMARY OF INVENTION

The present inventors applied metal-organic framework (MOF) mediated biomimetic mineralisation technique for lipid-nucleic acid molecule complex encapsulation and investigated the efficiency and efficacy of the resulting formulation thereof. And surprisingly demonstrated that MOF encapsulation of a lipid-nucleic acid molecule complex stabilises the lipid-nucleic acid molecule complex for long term storage without freezing whilst retaining transfection potential.

Accordingly, the present disclosure provides a stabilized composition comprising a lipid-nucleic acid molecule complex encapsulated within a Metal Organic Framework (MOF) shell. The lipid-nucleic acid molecule complex does not localise within the MOF pores owing to its size but is encapsulated by a MOF shell that forms around the lipid-nucleic acid complex.

In one embodiment, the nucleic acid molecule is a DNA, RNA, oligonucleotide, antisense, or siRNA molecule. For example, the nucleic acid molecule may be a mRNA vaccine. The mRNA vaccine may be replication competent or incompetent. In one embodiment, the nucleic acid may be a vector or a plasmid.

Advantageously, the compositions of the disclosure maintain the physical stability and/or chemical stability and/or biological activity of the lipid-nucleic acid molecule complex. In one embodiment, the composition is characterized as having improved stability over 4 weeks as compared to a comparative composition comprising the lipid-nucleic acid molecule complex without the outer protective MOF shell. For example, the composition maintains at least 50%, at least 55%, at least 60%, at least 70%, at least 75% of its activity after 4 weeks of storage at temperatures up to 37° C. Preferably, the composition maintains at least 50% of its activity for up to 12 weeks. More preferably, the composition maintains at least 50% of its activity after 12 weeks of storage at temperatures up to 37° C. In some embodiments the composition is stored between 4 to 37° C. In one embodiment, the composition is stored at 4° C. The structural integrity of the lipid-nucleic acid molecule complex may be determined by TEM imaging for example whilst its functional integrity may be demonstrated by retention of transfection/transformation potential, or its ability to deliver payload (nucleic acid to generate protein for an immune response).

Any biocompatible MOF can be used. In some embodiments, the MOF is based on a carboxylic acid-based precursor ligand (e.g., a lactic acid based MOF), a dicarboxylate acid based ligand (e.g., fumaric acid, succinic acid, or malic acid based MOF), a tricarboxylate ligand (e.g. benzene tricarboxylate based MOF), an imidazolate ligand (ZIFs) and other organic molecules where for example, peptides or small molecules may be used as ligands to make the MOF biocompatible.

In one embodiment, the MOF is a zeolitic imidazolate framework (ZIF), for example, ZIF-8, ZIF-10, ZIF-90, ZIF-C or ZIF-L. In one embodiment the ZIF is ZIF-8. In another embodiment, the MOF is an aluminium based MOF, for example, aluminium fumarate, aluminium-cyclodextrin (alpha, beta, gamma), aluminium-tartrate, aluminium-gallate, MIL-53(Al), MIL-118A(Al), CAU-23(Al), MIL-96, MIL-100, MIL-101, NH-MIL-101, MIL-101-NH2, MIL-68, CAU-1, CAU-1-OH, CAU-10, CAU-23, MIL-69, MIL-116, MIL-118, MIL-120, MIL-121, MIL-122 and MIL-160. CAU-3, CAU-4, CAU-6, CAU-8, CAU-11, CAU-12, CAU-13, and CAU-15. BIT-72, BIT-73, BIT-74, KMF-1, PCN-333, MOF-253, DUT-5, 467-MOF, or AL-MIL-53-NH2. In one embodiment, the MOF is aluminium fumarate. The MOF may be crystalline, amorphous, or mixed phase.

In one embodiment, the composition is dried, for example, air dried, vacuum-dried, or freeze dried. In one or a further embodiment, the composition comprises one or more excipients, for example, a disaccharide such as sucrose or trehalose, lyophilized skim milk, BSA, serum, or mannitol. Such excipients may protect the MOF shell during drying.

The present disclosure also provides a method for producing a stabilized composition, the method comprising:
a. providing a lipid-nucleic acid molecule complex;
b. providing a ligand precursor;
c. providing a metal salt;
d. reacting the lipid-nucleic acid molecule complex, the ligand precursor and the metal salt to form a metal organic framework shell encapsulating the lipid-nucleic acid molecule complex.

Advantageously, the present disclosure provides a universal approach for the thermal stabilisation of lipid-nucleic acid molecule complexes based on MOF material encapsulation.

In one embodiment, the lipid nucleic acid molecule complex comprises at least one or more of an ionizable lipid (e.g., 4-hydroxybutyl)azanediyl]di(hexane-6,1-diyl) bis(2-hexyldecanoate (ALC-0315) (Acuitas Therapeutics)), helper/neutral lipid (e.g., DSPC 1,2-distearoyl-sn-glycero-3-phosphocholine), cholesterol and PEGylated lipid (e.g., 2-(polyethylene glycol)-2000]-N,N-ditetradecylacetamide (ALC-0159)).

In some embodiments when using an ionizable lipid, the lipids are present in the molar ratio of ionizable lipid and helper/neutral lipid:stabilisation lipid (e.g., cholesterol and or PEGylated lipid) of >50%:<50%.

In one embodiment, the lipid-nucleic acid molecule complex is in a buffered solution. In one example, the pH of the buffered solution is neutral (e.g., about 7 to about 7.4, for example 7.4).

In some embodiments, the method further comprising preparing the lipid-nucleic acid molecule complex. In one embodiment, preparing the lipid-nucleic acid molecule complex comprises:
a. reacting the lipid and nucleic acid molecule to form the lipid-nucleic acid molecule complex.

In one embodiment, the lipid comprises at least one or more of an ionizable lipid (e.g., 4-hydroxybutyl)azanediyl]di(hexane-6,1-diyl) bis(2-hexyldecanoate (ALC-0315) (Acuitas Therapeutics))), helper/neutral lipid (e.g., DSPC 1,2-distearoyl-sn-glycero-3-phosphocholine), cholesterol and PEGylated lipid (e.g., 2-(polyethylene glycol)-2000]-N,N-ditetradecylacetamide (ALC-0159)).

In some embodiments when using an ionizable lipid, the molar ratio of ionizable lipid and helper/neutral lipid:stabilisation lipid (e.g., cholesterol and or PEGylated lipid) of >50%:<50%. For example, the molar ratio of ionizable lipid:helper/neutral lipid:cholesterol:PEGylated lipid may be 46.3:9.4:42.7:1.6

The present inventors have also shown that the N:P ratio of the lipid-nucleic acid molecule complex influences the transfection efficiency of the nucleic acid.

In some embodiments, the lipid and nucleic acid molecule are reacted at a ratio of positively chargeable polymer amine (N=nitrogen) groups to negatively charged nucleic acid phosphate (P) groups of about 2, for example, about 3, about 4, about 5, about 6, about 7, about 8, about 9, or about 10. In one example, the ratio of positively chargeable polymer amine (N=nitrogen) groups to negatively charged nucleic acid phosphate (P) groups is between about 4 and about 10, for example, between about 4 and about 8, for example, between about 4 and about 6. In another example, the ratio of positively chargeable polymer amine (N=nitrogen) groups to negatively charged nucleic acid phosphate (P) groups is about 6.

In some embodiments a low pH buffer may be used to ionize the lipid and enable its electrostatic interaction with the mRNA to form the lipid-nucleic acid molecule complex. Preferably, this pH is subsequently raised (e.g. by dialysis) to promote subsequent MOF formation. In some embodiments, the pH is raised prior to the addition of the ligand precursor and/or metal salt. In some embodiments, the pH is neutral (e.g. about 7) or slightly higher (e.g., about 7.4).

In one embodiment, preparing the lipid-nucleic acid molecule complex comprises:
a. reacting the lipid and nucleic acid molecule at low pH (e.g., at a pH of between about 3 to less than about 7, for example at a pH of between about 3 and about 5, for example at a pH of 4) to form a lipid-nucleic acid molecule complex;
b. adjusting the pH to a neutral pH (e.g., about 7 to about 7.4, for example 7.4) (e.g., using a buffer system such as sodium citrate buffer).

Advantageously, adjusting the pH prior to providing a ligand precursor and/or providing a metal salt improves yield of the MOF encapsulated lipid-nucleic acid molecule complex.

In one embodiment, one or more of the lipid-nucleic acid molecule complex, the ligand precursor and the metal salt are provided in solution in one or mixed solvents, for example, water, alcohol, or other organic solvent, or buffer, or cell culture medium, or reduced serum cell culture medium such as OptiMEM™.

In one or a further embodiment, the solution comprises one or more excipients, for example, a disaccharide such as sucrose or trehalose, lyophilized skim milk, BSA, serum, or mannitol.

In one or a further embodiment, the ligand precursor is 2-methylimidazole, for example, 80 to 640 mM 2-methylimidazole in buffer or cell culture medium. In another embodiment, the ligand precursor is fumaric acid, for example, 5 to 45 mM fumaric acid in buffer or cell culture medium.

In one or a further embodiment, the metal salt is zinc acetate, for example, 20 to 160 mM zinc acetate dihydrate in buffer or cell culture medium. In another embodiment, the metal salt is sodium aluminate, for example, 5 to 45 mM sodium aluminate in buffer or cell culture medium.

In one embodiment, the metal salt and ligand precursor is provided at a ratio from 100:1 to 1:100. For example, where the metal salt is zinc acetate and the ligand precursor is 2-methylimidazole, the metal salt:ligand precursor ratio is preferably between 1:4 and 1:8. In another example, the metal salt is sodium aluminate and the ligand precursor is fumaric acid and the metal salt:ligand precursor ratio is 1:1.

In some embodiments, the lipid-nucleic acid molecule complex, the ligand precursor, and the metal salt solution are added simultaneously or sequentially. For example, the lipid-nucleic acid molecule complex is added to the ligand precursor, followed by the metal salt solution. In another example, the lipid-nucleic acid molecule complex is added to the metal salt solution, followed by the ligand precursor. The skilled person will appreciate that the order may depend on the pH of the MOF precursors. For example, fumaric acid is acidic and would preferably be added after the ligand precursor.

In one or a further embodiment, the lipid-nucleic acid molecule complex, the ligand precursor, and the metal salt solution are incubated for about 5 to 30 minutes.

In one or a further embodiment, the method further comprises centrifuging the reaction mixture of step (d) to pellet the metal organic framework encapsulating the lipid-nucleic acid molecule complex.

In one or a further embodiment, the method further comprises adding one or more excipients, for example, trehalose (for example, between 0.5-20% w/w solution such as 5-10% w/w solution), before the metal organic framework shell forms.

In one or a further embodiment, the pellet is collected.

In one or a further embodiment, the pellet is dried, for example, air dried, vacuum dried, or freeze dried. In one embodiment, the method further comprises adding one or more excipients, for example, lyophilized skim milk (or example, between 0.5-20% w/w solution such as 5-10% w/w solution), prior to drying.

The stabilized lipid-nucleic acid molecule complex composition may be administered to a subject as part of a vaccination strategy. In one embodiment the subject is a mammal, for example an human. In another embodiment, the subject is avian, for example, a chicken. The stabilized lipid-nucleic acid molecule complex may also be administered to a subject for gene therapy or drug therapy purposes. In one embodiment, the composition is administered as is (i.e., with the outer protective MOF shell). In an alternate embodiment, the lipid-nucleic acid molecule complex is first released from the MOF shell.

Accordingly, the present disclosure also provides a method of preparation of the composition of the disclosure for administration, wherein the method comprises adding a release buffer, for example citrate buffer or an EDTA buffer, to the composition to chelate the metal ions causing MOF disintegration, and thereby release the lipid-nucleic acid molecule complex. In one embodiment, between about 10 mM to about 500 mM sodium citrate at a pH of about 5.0 is used, for example, 50 mM sodium citrate.

The present disclosure is not to be limited in scope by the specific examples described herein, which are intended for the purpose of exemplification only. Functionally-equivalent formulations, compositions and methods are clearly within the scope of the present disclosure.

Any example/embodiment of the present disclosure herein shall be taken to apply mutatis mutandis to any other example/embodiment of the disclosure unless specifically stated otherwise.

### DESCRIPTION OF THE DRAWINGS

FIG. 1. Thermal Stabilization of MOF encapsulated lipid-nucleic acid (plasmid green fluorescent protein DNA) complex. Figure shows the significant increase in thermal stability of MOF encapsulated liposomal complexes, MOF@lipo@pGFP(DNA) compared with lipo@pGFP(DNA) complexes without the MOF protection. FIG. 1a shows the transfection efficiency after the storage of the two complexes, MOF@lipo@pGFP and lipo@pGFP at −80° C., −20° C., 4° C., Room Temperature (R.T.) and 37° C. for 24 h against the efficiency of the freshly prepared complexes (control) and time t=0. FIG. 1b shows the storage stability of two complexes at 7 d (1 week) at the aforementioned temperature conditions. FIG. 1c shows representational fluorescent microscopy images from the test conditions in FIGS. 1a and 1b (scale=100 μm). FIG. 1d shows the transmission electron microscopy (TEM) images for (i) lipo@DNA and (ii) MOF@lipo@DNA complexes.

FIG. 2. The 24 h hour storage stability experiment as shown in FIG. 1a was repeated with removal of the complexes from the cells after 5 hours (and addition of culture medium), resulting in improved initial transfection efficiency. Transfection efficiency is expressed as number of cell or cell counts of GFP expressing cells as a percent of total cell confluence. The results demonstrate repetitive, significant increase thermal of in stability MOF encapsulated liposomal complexes, MOF@lipo@pGFP(DNA) compared with lipo@pGFP(DNA) complexes without the MOF protection after the storage of the two complexes at −80° C., −20° C., 4° C., Room Temperature (R.T.) and 37° C. for 24 h against the efficiency of the freshly prepared complexes (control) at time t=0.

FIG. 3. MOF encapsulation, release, and transfection efficiency of a MOF@lipo@EGFP(mRNA) complex. A series of transfection studies were performed with four different MOF precursor concentrations (A-D) encapsulating the lipo@mRNA complex. The studies were performed on two cell lines, Hela and A549. The transfection efficiency for MOF A and B was maintained after release of the MOF compared to the unencapsulated lipo@mRNA. MOF C and D appears to be detrimental to transfection efficiency due to excess MOF concentrations causing reduced cell viability.

FIG. 4. MOF encapsulation and thermal stabilization of a Lipo@EGFP mRNA (enhanced green fluorescent protein mRNA) complex. To demonstrate the ability for the MOF encapsulation to stabilise the lipo@mRNA complex, the encapsulated MOF@lipo@mRNA samples were stored at −80° C., −20° C., 4° C., Room Temperature (R.T.) and 37° C. for 24 h and 7 days. MOF B precursor concentration were used. Results show that the MOF encapsulated complexes have higher transfection after thermal challenges and storage at 4° C., Room Temperature (R.T.) and 37° C. after 24 h and 7 days.

FIG. 5. Synthesis and Transmission Electron Microscopy visualization of MOF Encapsulation and Release of the Lipid Nanoparticle (LNP) containing nucleic acid, EGFP (mRNA). The schematic illustrates the steps involved in the study; (1) LNP@nucleic acid is prepared using 4 lipid components and EGFP mRNA, (2) the LNP@mRNA is encapsulated with MOF, (3) the MOF shell is released with a buffer after storage and (4) the LNP@mRNA is ready for administration or transfection studies. The TEM images are a visualisation of each step in the schematic; (a) LNP@nucleic acid particles that were prepared using 4 lipid components and EGFP mRNA, (b) MOF@LNP@mRNA particles after encapsulated, (c) partial release of the MOF shell using a citrate buffer and (d) the LNP@mRNA particles after release of the MOF, showing that they remain intact.

FIG. 6. MOF encapsulation and stabilization of a lipid nanoparticle-mRNA formulation, MOF@LNP@mRNA. To demonstrate the ability for the MOF encapsulation to stabilise the 4 lipid component LNP@mRNA complex, the encapsulated MOF@LNP@mRNA samples were stored at −80° C., 4° C., Room Temperature (R.T.) and 37° C. for 14 days. Results indicate that the MOF encapsulated LNP@mRNA had greater transfection efficiency of the EGFP after encapsulation, storage and release.

### DETAILED DESCRIPTION

Before describing the present invention in detail, it is to be understood that the terminology used herein is for the purpose of describing particular embodiments only and is not intended to be limiting.

As used in this specification and the appended claims, the singular forms “a”, “an” and “the” include plural referents unless the content clearly dictates otherwise. Thus, for example, reference to a metal salt, a ligand precursor includes a combination of two or more such molecules.

Throughout the description and claims of the specification, the word “comprise” and variations of the word, such as “comprising” and “comprises”, is not intended to exclude other additives, components, integers or steps.

The term “about”, as used herein, indicates the value of a given quantity can include quantities ranging within 10% of the stated value, or optionally within 5% of the value, or in some embodiments within 1% of the value.

As used herein “@” denotes that the substance recited before the “@” symbol encapsulates, forms a complex with or is mixed or dispersed with the substance recited after the “@” symbol. For example, “MOF@lipid-nucleic acid complex” is intended to refer to a MOF encapsulated lipid-nucleic acid complex, lipo@mRNA is intended to refer to a mRNA encapsulated, complexed, mixed or dispersed with a liposome, MOF@lipo@mRNA is intended to refer to a MOF encapsulated liposome-mRNA complex. For the avoidance of doubt, where MOF is mentioned, the MOF is always on the outer layer, encapsulating the substance after the @, forming a MOF shell around it.

Unless specifically defined otherwise, all technical and scientific terms used herein shall be taken to have the same meaning as commonly understood by one of ordinary skill in the art

All publications or patents cited herein are entirely incorporated herein by reference. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987 including all updates until present); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994 including all updates until present); Colligan et al., Current Protocols in Protein Science, John Wiley & Sons, NY, N.Y., (1997 including all updates until present).

A reference herein to a patent document or other matter which is given as prior art is not to be taken as admission that the document or matter was known or that the information it contains was part of the common general knowledge as at the priority date of any of the claims.

As used herein “Metal-Organic Frameworks (MOFs)” are one-two or three dimensional organic-inorganic hybrid coordination networks composed of metal ions or clusters (termed secondary binding units (SBUs) bridged by organic ligands. The organic ligands may be carboxylates, or anions, such as phosphonate, sulfonate, and heterocyclic compounds. The organic ligands are C-2 to C-30 organic compounds, composed of substituted or unsubstituted, straight chain or cyclic organic molecules, substituted or unsubstituted aromatic compounds, or substituted or unsubstituted heteroaromatic compounds. The ligands maybe biocompatible peptides or small organic molecules. The organic ligands do not contain functionalised organic block polymers or copolymers as linkers between metal atoms. The organic ligands do not also contain side chains of organic block polymers or copolymers.

The geometry is determined by the coordination number, coordination geometry of the metal ions, and the nature of the functional groups. A variety of SBU geometries with different number of points of extension such as octahedron (six points), trigonal prism (six points), square paddle-wheel (four points), and triangle (three points) have been observed in MOF structures. The final frame-work topology of MOF is governed by both SBU connectors and organic ligands. MOFs may have pore openings up to 2 nm size (microporous) or may have a pore size of 2-50 nm (mesoporous). The MOF may be non-porous. The synthesis of MOFs involves reaction conditions and simple methods such as solvothermal, ionothermal, diffusion, microwave methods, ultrasound-assisted, template-directed syntheses, and others. Various MOFs composed of different metal ions and organic ligands have been described. Particularly useful MOFs in the stabilisation of lipid-nucleic acid molecule complexes of the disclosure are biocompatible. Such MOFs may be synthesized from non-toxic cations such as calcium, iron, zinc, aluminium, molybdenum, sodium, copper, potassium and magnesium. The MOF may be amorphous, or crystalline, or a mixed phase. The skilled person will recognise that the terms “coordination polymers” and “Metal-Organic Frameworks (MOFs)” are used interchangeably in the art.

As used herein, “MOF shell” refers to a MOF layer that encapsulates the lipid-nucleic acid molecule complex to form a protective coating for the storage of the lipid-nucleic acid molecule complex. The lipid-nucleic acid molecule complex does not localise within the MOF pores owing to its size.

As used herein, “zeolitic imidazolate framework” (or “ZIF”) refer to microporous structures having frameworks commonly found in zeolites and/or in other crystalline materials wherein each vertex of the framework structure is comprised of a single metal ion and each pair of connected adjacent vertices of the framework structure is linked by nitrogen atoms of an imidazolate anion or its derivative as the ligand. ZIFs are a subset of MOFs. The frameworks can comprise any of the networks defined in the Atlas of Zeolite Structure Types and the Reticular Chemistry Structure Resource (RCSR) Database known in the literature. Particularly useful ZIFs in the stabilisation of lipid-nucleic acid molecule complexes of the disclosure are biocompatible. Such ZIFs may be synthesized from non-toxic cations such as calcium, iron, zinc, aluminium, molybdenum, sodium, copper, potassium and magnesium. The ZIF may be amorphous, or crystalline, or a mixed phase. In one embodiment, the ZIF is amorphous.

A “stable” formulation or composition is one in which the lipid-nucleic acid molecule complex therein essentially maintains its physical stability (or structural integrity) and/or chemical stability and/or biological activity upon storage and on release from the MOF shell. For example, the lipid-nucleic acid molecule complex maintains its transfection capacity and/or its ability to deliver payload (e.g., nucleic acid). As used herein “maintains” will be understood to include partial retention, for example, at least 60%, at least 70%, at least 75%, at least 80%, at least 85%, at least 90%, at least 95% of its activity prior to encapsulation within a MOF shell. Stability can be measured at a selected temperature for a selected period. Trend analysis can be used to estimate an expected shelf life before a material has actually been in storage for that time period.

As used herein, the term “ambient room temperature” refers to typical controlled indoor temperatures, such as from about 16° C. to about 27° C., or more typically from about 18° ° C. to about 25° C., and often about 24° C.

As used herein, the term “dry” or “dried” in reference to the lipid-nucleic acid molecule complex formulations described herein refers to a composition from which a substantial portion of any water has been removed to produce a de-hydrated phase of the composition. The term does not require the complete absence of moisture. The lipid-nucleic acid molecule complex compositions described herein generally have a moisture content from about 0.1% by weight and about 25% by weight.

### MOF-Encapsulated Lipid-Nucleic Acid Molecule Complexes

The lipid-nucleic acid molecule complex compositions of the disclosure are encapsulated by a MOF protective shell comprising metal ions or clusters coordinated to organic ligands.

Suitable metal ions can be selected from Group 1 through 16 metals of the IUPAC Periodic Table of the Elements including actinides, and lanthanides, and combinations thereof. The metal ion may be selected from Na⁺, K⁺, Mg²⁺, Ca²⁺, Mo⁶⁺, Mo³⁺, Fe³⁺, Fe²⁺, Cu²⁺, Cu⁺, Zn²⁺, Al³⁺, and combinations thereof.

Suitable metal ion coordinating organic ligands can be derived from oxalic acid, malonic acid, succinic acid, glutaric acid, phthalic acid, isopthalic acid, terephthalic acid, citric acid, trimesic acid, 1,2,3-triazole, pyrrodiazole, imidazole or squaric acid.

Metal ions and organic ligands used to construct MOF encapsulated lipid-nucleic acid molecule complexes with good biocompatibility are preferred. The skilled person will appreciate that it will be preferable to use non-toxic MOF precursors so as to be useful in therapeutic settings, and to minimise cell death in in vitro, ex-vivo applications. Metal ions with good biocompatibility include, for example, sodium, potassium, calcium, iron, zinc, copper, zirconium, titanium, magnesium, manganese, molybdenum, molybdenum or aluminium. Preferably the metal ions are selected from the list consisting of Na⁺, K⁺, Mg²⁺, Ca²⁺, Mo⁶⁺, Mo³⁺, Fe³⁺, Fe²⁺, Cu²⁺, Cu⁺, Zn²⁺, Al³⁺, Zr⁴⁺, Mo⁶⁺, Mo³⁺, Ag¹⁺, Ti³⁺, Ti⁴⁺, Ta⁵⁺, or combinations thereof.

In some embodiments, MOFs are selected from mixed component MOFs, known as MC-MOFs. MC-MOFs have a structure that is characterised by more than one kind of organic ligand and/or metal. MC-MOFs can be obtained by using different organic ligands and/or metals directly in the solution into which MOF precursors and nucleic acid are combined, or by post-synthesis substitution of organic ligands and/or metals species of formed MOFs.

In some embodiments, the MOF is a zinc imidazolate framework (ZIF). ZIFs are a sub-class of MOFs that particularly suited to biologic applications. ZIF frameworks feature tetrahedrally-coordinated transition metal ions (e.g., Fe, Co, Cu, Zn) connected by organic imidazolate organic ligands, resulting in three-dimensional porous solids.

Accordingly, MOFs that may be made in accordance with the invention may be carboxylate-based MOFs, heterocyclic azolate-based MOFs, metal-cyanide MOFs. Specific examples of MOFs that may be made according to the present invention include those commonly known in the art as CD-MOF-1, CD-MOF-2, CD-MOF-3, CPM-13, FJI-1, FMOF-1, HKUST-1, IRMOF-1, IRMOF-2, IRMOF-3, IRMOF-6, IRMOF-8, IRMOF-9, IRMOF-13, IRMOF-20, MIL-101, MIL-125, MIL-53, MIL-88 (including MIL-88A, MIL-88B, MIL-88C, MIL-99 D series), MOF-5, MOF-74, MOF-177, MOF-210, MOF-200, MOF-205, MOF-505, MOROF-2, MOROF-1, NOTT-100, NOTT-101, NOTT-102, NOTT-103, NOTT-105, NOTT-106, NOTT-107, NOTT-109, NOTT-110, NOTT-111, NOTT-112, MOTT-113, NOTT-114, NOTT-140, NU-1000, rho-ZMOF, PCN-6, PCN-6′, PCN9, PCN10, PCN12, PCN12′, PCNI4, PCN16, PON-17, PCN-21, PCN46, PCN66, PCN68, PMOF-2(Cu), PMOF-3, SNU-5, SNU-15, SNU-215, SNU-21H, SNU-50, SNU-77H, UiO-66, UIO-67, TUDMOF-1, UMCM-2, UMCM-150, UTSA-20, ZIF-2, ZIF-3, ZIF-4, ZIF-8, ZIF-9, ZIF-10, ZIF-11, ZIF-12, ZIF-14, ZIF-20, ZIF-21, ZIF-23, ZIF-60, ZIF-61, ZIF-62, Z1F-64, Z1F-65, ZIF-67, ZIF-68, ZIF-69, ZIF-70, ZIF-71, ZIF-72, ZIF-73, ZIF-74, ZIF-75, ZIF-76, ZIF-77, or ZIF-90.

The MOF-encapsulated lipid-nucleic acid molecule complex compositions provided herein advantageously may be characterized as having improved stability. As used herein, “improved stability” of a lipid-nucleic acid molecule complex composition may be determined by using a transfection assay after storage for a given time and temperature.

For example, the lipid-nucleic acid molecule complex composition may be characterized as having improved stability in the composition over one month as compared to a comparative composition comprising the lipid-nucleic acid molecule complex without MOF encapsulation, over three months as compared to such a comparative composition, over six months as compared to such a comparative composition, over nine months as compared to such a comparative composition, or over one year as compared to such a comparative composition.

In some embodiments, the stability of the composition is shown by the relative activity of the lipid-nucleic acid molecule complex after storage at room temperature or at elevated temperatures of up to 40° C. as compared to the initial activity of the lipid-nucleic acid molecule complex. For example, the stability of the composition may be characterized by the lipid-nucleic acid molecule complex maintaining at least 50% of its activity after three months of storage at temperatures up to 40° C., at least 60% of its activity after three months of storage at temperatures up to 40° C., at least 70% of its activity after three months of storage at temperatures up to 40° C., at least 75% of its activity after three months of storage at temperatures up to 40° C., at least 80% of its activity after three months of storage at temperatures up to 40° C., or at least 90% of its activity after three months of storage at temperatures up to 40° C.

In some embodiments, the stability of the composition may be characterized by the lipid-nucleic acid molecule complex maintaining at least 50% of its activity after three months of storage at 37° C., at least 60% of its activity after three months of storage at 37° C., at least 70% of its activity after three months of storage at 37° C., at least 75% of its activity after three months of storage at 37° C., at least 80% of its activity after three months of storage at 37° C., or at least 90% of its activity after three months of storage at 37° C.

### Methods of Manufacture

The lipid-nucleic acid molecule complex formulations described herein are generally prepared by biomimetic mineralization of the lipid-nucleic acid molecule complex to encapsulate it within a MOF shell.

The method of the invention comprises combining in a solution the lipid-nucleic acid molecule complex and MOF precursors.

MOF precursors include those compounds known in the art that provide the metal ions described herein in the solution within a suitable solvent. Those compounds may be salts of the relevant metal ions, including metal hydroxides, chlorides, oxychlorides, -nitrates, oxynitrates, -acetates, acetoacetates, -sulphates, trisulphates, hydrogen sulphates, -bromides, -carbonates, -phosphates, and derivatives thereof, including mono- and poly-hydrate derivatives.

Examples of suitable metal salt precursors include, but are not limited to, zinc nitrate (Zn(NO₃)₂·xH₂O), iron(III) nitrate (Fe(NO₃)₃·XH₂O), aluminium nitrate (AI(NO₃)₃·xH₂O), magnesium nitrate (Mg(NO₃)₂ xH₂O), calcium nitrate (Ca(NO₃)₂ xH₂O), gadolinium nitrate (Gd(NO₃)₃·xH₂O), zinc chloride (ZnCl₂ xH₂O), iron(III) chloride (FeCl₂ xH₂O), iron(II) chloride (FeCl₂·xH₂O), aluminium chloride (AlCl₃·xH₂O), magnesium chloride (MgCl₂·xH₂O), calcium chloride (CaCl₂·xH₂O), gadolinium chloride (GdCl₃·xH₂O), zinc acetate (Zn(CH₃COO)₂·xH₂O), iron(III) acetate (Fe(CH₃COO)₃·xH₂O), iron(O) acetate (Fe(CH₃COO)₂·xH₂O), aluminium acetate (Al(CH₃COO)₃·xH₂O), magnesium acetate (Mg(CH₃COO)₂·xH₂O), calcium acetate (Ca(CH₃COO)₂·xH₂O), gadolinium acetate (Gd(CH₃COO)₃ xH2O), zinc sulphate (ZnSO₄·xH₂O), iron(III) sulphate (Fez(SO₄)₃·xH₂O), iron(II) sulphate (FeSO₄·xH₂O), aluminium sulphate (Al₂(SO₄)₃·xH₂O), magnesium sulphate (MgSO₄·xH₂O), calcium sulphate (CaSO₄·xH₂O), zinc hydroxide (Zn(OH)₂ xH₂O), iron(III) hydroxide (Fe(OH)₃·xH₂O), iron(III) oxide:hydroxide (FeO(OH) xH₂O), iron(II) hydroxide (Fe(OH)₂·xH₂O), aluminium hydroxide (Al(OH)₃ xH₂O), magnesium hydroxide (Mg(OH)₂ xH₂O), calcium hydroxide (Ca(OH)₂ xH₂O), zinc bromide (ZnBr₂·xH2O), iron(III) bromide (FeBr₃ xH₂O), iron(II) bromide (FeBr₂ xH₂O), aluminium bromide (AlBr₃ xH₂O), magnesium bromide (MgBr₂·xH₂O), calcium bromide (CaBr₂·H₂O), gadolinium bromide (GdBr₃·xH₂O), zinc carbonate (ZnCo₃·xH₂O), iron(III) carbonate (Fez(CO₃)₃·xH₂O), aluminium carbonate (Al₂(CO₃)₃·xH₂O), magnesium carbonate (MgCO₃·xH₂O), calcium carbonate (CaCO₃·xH₂O), gadolinium carbonate (Gd₂(CO₃)₃·xH₂O), zirconium(IV) nitrate (Zr(NO₃)₄·xH₂O), zirconium(IV) chloride (ZrCl₄ xH₂O), zirconium(IV) acetate (C₄H₆O₄Zr·xH₂O); silver(I) nitrate (AgNO₃ xH₂O), silver(I) nitrite (AgNO₂ xH₂O), silver(I) chloride (AgCl·xH₂O), silver(I) acetate (AgC₂H₃O₂·xH₂O); titanium(IV) tetrachloride (TiCl₄ xH₂O), titanium(III) trichloride (TiCl3₄ xH₂O), titanium nitrate(IV) (Ti(NO₃)₄ xH₂O), titanium(IV) bromide (Br₄Ti xH₂O); tantalum chloride (TaCl₅ xH₂O), and mixtures thereof, where x ranges range from 0 to 12.

MOF precursors also include organic ligands of the kind described herein that coordinate the metal ion clusters in the MOF framework. The organic ligands include molecules that have at least two chemical moieties capable of coordinating a metal ion. In some embodiments, these groups comprise carboxylates, phosphonates, sulphonates, N-heterocyclic groups, and combinations thereof.

Examples of organic ligand precursors include, but are not limited to, α-cyclodextrin, β-cyclodextrin, γ-cyclodextrin, oxalic acid, oxalate, fumaric acid, fumarate, maleic acid, maleate, 4,4,4″-[benzene-1,3,5-triyl-tris(ethyne-2,1-diyl)]tribenzoate, biphenyl-4,4′-dicarboxylate, 4,4,4″-[benzene-1,3,5-triyl-tris(benzene-4,1-diyl)]tribenzoate, 1,3,5-benzenetribenzoate, 1,4-benzenedicarboxylate, benzene-1,3,5-tris(1H-tetrazole), 1,3,5-benzenedicarboxylic acid, terephthalic acid, imidazole, benzimidazole, 2-nitroimidazole, 2-methylimidazole (Hmlm), 2-ethylimidazole, 5-chloro benzimidazole, purine, fumaric acid, 1,4-Bis(1-imidazolyl)benzene), 4,4,-Bispyridyl, 1,4-Diazabicyclo[2,2,2]octane, 2-amino-1,4-benzenedicarboxylic, 2-amino-1,4-benzenedicarboxylic acid, 4,4′-Azobenzenedicarboxylate, 4,4′-Azobenzenedicarboxylic acid, Aniline-2,4,6-tribenzoate, Amiline-2,4,6-tribenzic acid, Biphenyl-4,4′-dicarboxylic acid, 1,1′-Biphenyl-2,2′,6,6′-tetracarboxylate, 1,1′-Biphenyl-2,2′,6,6′-tetracarboxylic acid, 2,2′-Bipyridyl-5,5′-dicarboxylate, 2,2-Bipyridyl-5,5′-dicarboxylic acid, 1,3,5-Tris(4-carboxyphenyl)benzene, 1,3,5-Tris(4-carboxylatephenyl)benzene, 1,3,5-Benzenetricarboxylate, 2,5-Dihydroxy-1,4-benzenedicarboxylate, 2,5-Dihydroxy-1,4-benzenedicarboxylic acid, 2,5-Dirnethoxy-1,4-benzenediearbOxylate, 2,5-Dimetboxy-1,4-benzenedicarboxylic acid, 1,4-Naphthalenedicarboxylate, 1,4-Naphthalenedicarboxylic acid, 1,3-Naphthalenedicarboxylate, 1,3-Naphthalenedicarboxyluc, acid, 1,7-Naphthalenedicarboxylate, 1,7-Naphthalenedicarboxylic acid, 2,6-Naphthalenedicarboxylate, 2,6-Naphthalenedicarboxylic acid, 1,5-Naphthalenedicarboxylate, 1,5-Naphthalenedicarboxylic acid, 2,7-Naphthalenedicarboxylate, 2,7-Naphthalenedicarboxylic acid, 4,4′,4′-Nitrilotrisbenzoate, 4,4′,4″-Nitrilotrisbenzoic acid, 2,4,6-Tris(2,5-dicarboxylphenylamino)-1,3,5-triazine, 2,4,6-Tris(2,5-dicarboxylatephenylamino)-1,3,5-triazine, 1,3,6.8-Tetrakis(4-carboxyphenyl)pyrene, 1,3,6,8-Tetrakis(4-carboxylatephenyl)pyrene, 1,2,4,5-Tetrakis(4-carboxyphenyl)benezene, 1,2,4,5-Tetrakis(4-carboxylatephenyl)benzene, 5,10,15,20-Tetrakis(4-carboxyphenyl)porphyrin, 5,10,15,20-Tetrakis(4-carboxylatephenyl)porphyrin, adenine, adeninate, fumarate, 1,2,4,5-benzenetetracarboxylate, 1,4,5-benzenetetracarboxylic acid, 1,3,5-benzenetribenzoic acid, 3,-amino-1,5-benzenedicarboxylic acid, 3-amino-1,5-benzenedicarboxylate, 1,3-benzenedicarboxylic acid, 1,3-benzenedicarboxylate, 4′,4′,4″-[benzene-1,3,5-triyl-tris(ethyne-2,1-diyl)]tribenzoic acid, 4,4′,4″-[benzene-1,3,5-triyl-tris(benzene-4,1-diyl)]tribenzoic acid, trans.trans-muconic acid, trans, trans-muconate, cis, trans-muconic acid, cis,trans-muconate, cis,cis-muconic acid, cis,cis-muconate, pyrazole, 2,5-dimethylpyrazole, 1,2,4-triazole, 3,5-dimethyl-1,2,4-triazole, pyrazine, 2,5-dimethylpyrazine, hexamethylentetraamine, nicotinic acid, nicotinate, isonicotinic acid, isonicotinate, 4-(3,5-dimethyl-1H-pyrazole)-benzoic acid, 2,5-furandicarboxylic acid, 2,5-furandicarboxylate, 3,5-dimethyl-4-carboxypyrazole, 3,5-dimethyl-4-carboxylatepyrazole, 4-(3,5-dimethyl-1H-pyrazol-4-yl)-benzoic acid, 4-(3,5-dimethyl-1H-pyrazol-4-yl)-benzoate, and mixtures thereof.

It will be understood that the organic ligands can also be functionalised organic ligands. For example, any one of the organic ligands described herein may be additionally functionalised by amino-, such as 2-aminoterephthalic acid, urethane acetamide-, or amide-. The organic ligand can be functionalised before being used as precursor for MOF formation, or alternatively the assembled MOF itself can be chemically treated to functionalise its bridging organic ligands.

A skilled person will be aware of suitable chemical protocols that allow functionalizing MOFs with functional groups, either by pre-functionalizing organic ligands used to synthesize MOFs or by post-functionalizing pre-formed MOFs.

Suitable functional groups that may be provided on the MOF include —NHR, —N(R)₂, —NH₂, —NO₂, —NH(aryl), halides, aryl aralkyl, alkehyl, alkynyl, pyridyl, bipyridyl, terpyridyl, anilino, —O(alkyl), cycloalkyl, cycloalkenyl., cycloalkynyl, sulfonamide, hydroxyl, cyano, —(CO)R, —(SO₂)R, —(CO₂)R, —SH, —S(alkyl), —SO₃H, —SO₃₋, M₊, —COOH, COO-M₊, —PO₃H₂, —PO₃H-M⁺, —PO₃²⁻M²⁺, —CO₂H, silyl derivatives., borane derivatives, ferrocenes and other metallocenes, where M is a metal atom, and R is C₁₋₁₀ alkyl.

There are no particular restrictions on the solvents that can be used to prepare the solution in which MOF precursors and lipid-nucleic acid molecule complex are combined, provided that (i) the MOF precursors are soluble in the solvent, and (ii) the lipid-nucleic acid molecule complex is compatible with the solvent. That is, the solvent will typically be one that does not adversely affect the physical stability and/or chemical stability and/or biological activity of the nucleic acid molecule and/or the lipid-nucleic acid molecule complex. Preferably the solvent, is biocompatible.

Examples of solvent that may be used include methanol, ethanol, dimethyl sulfoxide (DMSO), acetone, water and mixtures thereof.

In some embodiments, the solution into which the lipid-nucleic acid molecule complex and MOF precursors are combined is an aqueous solution, for example deionised water, or a physiological buffered solution (water comprising one or more salts such as KH₂PO₄, NaHaPO⁺, K₂HPO₄, Na₂HPO₄, Na₃PO₄, K₃PO₄, NaCl, KCl, MgCl₂, CaCl₂, etc.), or a cell culture medium such as OptiMEM™.

Provided the MOF forms, there is no particular limitation regarding the concentration of MOF precursors present in the solution. The skilled person will appreciate that the minimum precursor concentration is determined by the self-assembly of MOF and is dependent on the MOF type. Also, the maximum precursor concentration is determined by the toxicity of the MOF precursors to the lipid-nucleic acid molecule complex being encapsulated. And its biocompatibility and environmental toxicity. This further depends on, for example, the type of lipid-nucleic acid molecule complex, whether it is released from the MOF or used intact to deliver the nucleic acid molecule into cells, etc. For example, the minimum exemplified working precursor concentration for a 3.25 μg of a nucleic acid in 200 μl volume was 16 mM 2-methylimidazole (Hmlm) and 4 mM ZnAc (ZIF-8). The maximum precursor concentration exemplified was 320:80, limited by the toxicity of MOF in in vitro assays to the Hela cells used.

Concentrations of MOF precursors in the solution can include a range between about 0.001 M and 1 M, between about 0.01 M and 0.5 M, between about 0.01 and 0.2 M, between about 0.02 M and 0.2 M, between about 0.02 M and 0.15 M, between about 0.05 M and 0.15 M between about 0.08 M and 0.16 M. The values refer to concentration of organic ligand as well as concentration of metal salt, relative to the total volume of the solution containing the MOF precursors and lipid-nucleic acid molecule complex.

The ratio between the concentration of organic ligands and the concentration of metal salts is not limited, provided the ratio is adequate for the formation of MOF promoted by the combination with the lipid-nucleic acid molecule complex in accordance to the disclosure. In some embodiments, the organic ligand to metal salt ratio may range from 100:1 to 1:100, from 60:1 to 1:60 (mol:mol), from 30:1 to 1:30, from 10:1 to 1:10, from 5:1 to 1:5, from 2.5:1 to 1:2.5, from 2:1 to 1:2, or from 1.5:1 to 1:1.5.

Suitable concentrations of nucleic acid molecule in the solution can include a range of between about 0.1 and 20 mg/mL, between about 0.15 and 10 mg/mL between about 0.15 and 7.5 mg/mL, between about 0.2 and 5 mg/mL, between about 0.25 and 5 mg/mL, between about 0.03 and 5 mg/mL, between about 0.025 and 3 mg/mL, between about 0.025 and 2.5 mg/mL, between about 0.025 and 2 mg ml, between about 0.025 and 1.5 mg/mL, or between about 0.025 and 1.25 mg/mL. In one embodiment, the concentration of the nucleic acid molecule is between about 1 and 3 mg/mL.

According to the method of the disclosure, the lipid-nucleic acid molecule complex (in for example, a buffer or medium such as OptiMEM™ reduced serum medium) promotes formation of the encapsulating MOF framework.

By the lipid-nucleic acid molecule complex ‘promotes’ formation of the encapsulating framework is meant the lipid-nucleic acid molecule complex per se causes, induces or triggers formation of the MOF framework upon combination with the MOF precursors in a solution. As a result of the lipid-nucleic acid molecule complex promoting formation of the framework, the MOF framework forms around the lipid-nucleic acid molecule complex to eventually encapsulate it within a MOF outer shell.

Without being limited to theory, it is believed the lipid-nucleic acid molecule complex induced formation of MOF may be related to the charge, hydrophilicity/hydrophobicity nature or chelating ability of the specific lipid-nucleic acid molecule complex. It is believed that formation of encapsulating MOF is facilitated by the lipid-nucleic acid molecule complex affinity towards MOF precursors arising, for example, from intermolecular hydrogen bonding and hydrophobic interactions.

The resulting increase in the local concentration (i.e., in the immediate surroundings of the lipid-nucleic acid molecule complex) of both metal cations (deriving from the dissolution of the metal salt precursor) and organic ligands would facilitate pre-nucleation clusters of MOF framework.

In some embodiments, combining the MOF precursors in solution with the lipid-nucleic acid molecule complex is surprisingly sufficient to cause formation of the MOF framework. There is no need to apply other factors or reagents to trigger formation of the MOF. For example, it is not necessary to apply heat to the solution as conventionally done in traditional solvothermal MOF synthesis methods (which typically require use of a heat source such as an oven, for example a microwave oven, a hot plate, or a heating mantel). For example, for ZIF-8, which is composed of 2-methylimidazole (Hmlm) and Zn2+, the MOF shell can self-assemble spontaneously in a buffered solution or cell cultured medium around the lipid-nucleic acid molecule complex (at low precursor concentrations) without the application of external energy such as heat, pressure or even additional time. At higher precursor concentrations, the precursors can precipitate with or without the presence of the lipid-nucleic acid molecule complex.

Accordingly, in some embodiments formation of the encapsulating framework is effected at a solution temperature that is lower than 75° C., 50° C., or 35° C. Thus, the solution temperature may be between 2° C. and 75° C., between 2° C. and 60° C., between 16° C. and 27° C., or between 18° C. and 25° C. The skilled person will appreciate that the temperature used is dependent on the MOF type and the heat sensitivity of the lipid-nucleic acid molecule complex.

In some embodiments, the method is performed at ambient room temperature. For example, for aluminium fumarate, which is composed of fumaric acid and sodium aluminate, the MOF shell can self-assemble around the lipid-nucleic acid molecule complex at ambient room temperature. Performing the method at ambient room temperature or below is advantageous for heat sensitive lipid-nucleic acid molecule complexes.

There is no particular limitation on the order in which the MOF precursors and the lipid-nucleic acid molecule complex may be combined into the solution.

For example, a solution containing a metal precursor may be first mixed with a solution comprising an organic ligand, and a separate solution comprising a lipid-nucleic acid molecule complex is subsequently introduced into the solution containing the metal salt and the organic ligand. Alternatively, a solution comprising a lipid-nucleic acid molecule complex and an organic ligand may be first prepared, and subsequently introduced into a separate solution comprising a metal precursor. Also, a solution comprising a lipid-nucleic acid molecule complex and a metal precursor may be first prepared, and subsequently introduced into a separate solution comprising an organic ligand. Still further, separate solutions each individually comprising a metal precursor, an organic ligand and a lipid-nucleic acid molecule complex, respectively, may be mixed together at the same time. In one embodiment, the lipid-nucleic acid molecule complex is introduced into a solution comprising the MOF precursors.

Formation of MOF shell according to the method of the invention is advantageously fast. Depending on the lipid-nucleic acid molecule complex used and the type of MOF precursors used, it has been found that upon bringing the lipid-nucleic acid molecule complex and the MOF precursors together in a solution MOF may form within about 1 second, 10 seconds, 1 minute, 10 minutes, 30 minutes, 60 minutes or 2 hours.

The resultant lipid-nucleic acid molecule complex encapsulated MOF solution may be dried at any suitable temperature and pressure conditions, which preferably are selected to maintain the physical stability and/or chemical stability and/or biological activity of the lipid-nucleic acid molecule complex. In a preferred embodiment, the aqueous solution is dried at an ambient temperature for a time sufficient to form the dehydrated form of the lipid-nucleic acid molecule complex composition. For example, the aqueous solution may be dried at ambient temperature for a period from about 30 minutes to about one week to form the dehydrated form of the lipid-nucleic acid molecule complex formulation, for example, from about 45 minutes to about one week, or from about one hour to about one week, or from about one hour to about one day.

In other embodiments, the aqueous solution may be vacuum-dried or dried using a combination of air-drying and vacuum-drying. Although various temperatures and humidity levels can be employed to dry the aqueous solution, the formulations preferably are dried at temperature from −80° C. to 60° C. (e.g., from 15° C. to about 45° C., from about 25° C. to about 45° C., or at about ambient temperature) and 0 to 10% relative humidity. In some embodiments, one or more excipients are added to the solution prior to drying, for example, one or more disaccharides such as sucrose and/or trehalose, lyophilized skim milk, BSA, serum, and/or mannitol. Such excipients may protect the MOF shell during drying.

After manufacture and prior to use, the lipid-nucleic acid molecule complex compositions are packaged and stored under refrigeration, for example, at temperatures from about 2° C. to about 8° C.; in a freezer, for example at temperatures between −20° C. and 0° C.; at ambient temperature; or at uncontrolled temperature, for example, up to 55° C. The lipid-nucleic acid molecule complex composition is preferably stored at between 4° C. and 37° C. The storage may be for the shelf life of the product or for a period less than the shelf life of the product. Monitors or other temperature indicators may be used to identify when the lipid-nucleic acid molecule complex composition has exceeded a permissible level of thermal exposure. Advantageously, the lipid-nucleic acid molecule complex compositions provided herein impart greater thermostability than previously existing formulations, thereby minimizing contamination, degradation, or loss of activity that can occur when the lipid-nucleic acid molecule complex compositions are exposed to variable temperatures. Thus, the storage temperature for the lipid-nucleic acid molecule complex compositions provided herein is less critical than for previously existing formulations.

The lipid-nucleic acid molecule complex encapsulated within the MOF framework may be released into a solvent by dissolving the MOF suspended within the solvent, for example, by inducing a variation of the pH of the solvent.

Examples of MOFs that may be used in applications based on pH-triggered release of the lipid-nucleic acid molecule complex include MOFs that are stable at certain pH values, but dissolve at certain other pH values. For example, the MOF may be stable above a threshold pH value. In that case there is no detectable release of the lipid-nucleic acid molecule complex into the solution within which the MOF is suspended. However, the MOF may dissolve when the pH drops below the threshold, resulting in the release of the lipid-nucleic acid molecule complex into the solution. For example, certain ZIFs are stable at extracellular pH (about 7.4), but dissolve when the pH drops below 6.5, for example, at intracellular pH (about 6).

In this context, the stability of a MOF in a solvent at a certain pH is determined in relation to the amount of metal ions released into the solvent by the MOF when dissolving. The concentration of metal ions in the solvent is determined by Inductively Coupled Plasma (ICP) performed before and after exposure of the MOF to that pH condition for 2 hours. A MOF will be deemed ‘stable’ if the measured concentration of metal ion in solution after 2 hours differs by less than of 15% from the initial value.

### Nucleic Acid Molecule

The nucleic acid may be DNA, including linear and plasmid DNAs; RNA, including mRNA, siRNA, CRISPR, saRNA, and microRNA; an oligonucleotide; or a polynucleotide.

The deliberate delivery or introduction of nucleic acids into eukaryotic cells is typically referred to as “transfection”. Transfection may also refer to other methods and cell types, although other terms are often preferred. Transformation is typically used to describe non-viral DNA transfer in bacteria and non-animal eukaryotic cells, including plant cells. Genetic material (such as supercoiled plasmid DNA or siRNA constructs), or even proteins such as antibodies, may be “transfected”. As used herein, “transfection” relates generally to delivery of a molecule into a cell including bacterial and non-animal eukaryotic cells.

The present disclosure includes use of vectors for manipulation or transfer of genetic constructs. In some embodiments, a vector is a nucleic acid molecule, for example, a DNA molecule, that can be used to artificially carry foreign genetic material; into another cell, where it can be replicated or expressed. A vector containing foreign nucleic acid is referred to as a “recombinant vector”. Examples of vectors include, but are not limited to, plasmids, viral vectors, cosmids, extrachromosomal elements, minichromosomes.

The vector is generally a DNA sequence that consists of an insert (transgene) and a larger sequence that serves as the “backbone” of the vector. The purpose of a vector which transfers genetic information to another cell is typically to isolate, multiply, or express the insert in the target cell. Vectors designed specifically for the expression of the transgene in the target cell are called “expression vectors”, and generally have a promoter sequence that drives expression of the transgene. Selection of appropriate vectors is within the knowledge of those having skill in the art.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, for example, in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter. A promoter can be operably linked to numerous nucleic acids, for example, through an internal ribosome entry site.

The molecule may be able to modulate the expression or activity of a cellular target. The term “cellular target” refers to any component of a cell. Non-limiting examples of cellular targets include DNA, RNA, a protein, an organelle, a lipid, or the cytoskeleton of a cell. Other examples include the lysosome, mitochondria, ribosome, nucleus, or the cell membrane.

In some embodiments, the molecule is siRNA. siRNA, or “Small Interfering RNA,” in general is a class of double-stranded RNA molecules, typically 20-25 base pairs in length. siRNA plays a role in the RNA interference (RNAi) pathway, where it interferes with the expression of specific genes with complementary nucleotide sequence. Thus, for example, siRNA may have a sequence that is antisense to a sequence within a target gene. siRNA also acts in RNAi-related pathways in some cases, e.g., as an antiviral mechanism or in shaping the chromatin structure of a genome. The siRNAs typically have a structure comprising a short (usually 21-bp) double-stranded RNA (dsRNA) with phosphorylated 5′ ends and hydroxylated 3′ ends with two overhanging nucleotides. siRNAs are typically produced by the Dicer enzyme reacting with various precursor RNAs. Those of ordinary skill in the art will be able to identify siRNAs, many of which have been catalogued in publicly accessible databases.

The nucleic acid molecule may encode an antigen. As used herein, “antigen” refers to a biomolecule capable of eliciting an immune response in a host. In some embodiments, the antigen elicits a protective immune response. The antigen may elicit a humoral or cellular immune response, or both.

As used herein, the term “immune response” is intended to include, but is not limited to, T and/or B cell responses, that is, cellular and/or humoral immune responses. In one embodiment, the claimed methods can be used to stimulate cytotoxic T cell responses. The claimed compositions can be used to stimulate both primary and secondary immune responses. The immune response of a subject can be determined by, for example, assaying antibody production, immune cell proliferation, the release of cytokines, the expression of cell surface markers, cytotoxicity, and the like.

### Lipid-Nucleic Acid Molecule Complex

The lipid-nucleic acid molecule complexes of the disclosure can be produced with methods well known in the art.

In some embodiments, the lipid-nucleic acid molecule complexes of the disclosure are lipid-based nanoparticles. The lipid-based nanoparticles may be selected from the group comprising liposomes, micelles, solid lipid nanoparticles (SLNs or LNPs), cubosomes, hexosomes, spunge phases, asymmetrical liposomes particles (ALPs) and stable nucleic acid lipid nanoparticles (SNALPs).

In one embodiment, the lipid-nucleic acid molecule complexes are liposomes.

Liposomes are composed of a lipid bilayer that forms in the shape of a hollow sphere encompassing an aqueous phase. As such, any cargo of interest can be encapsulated within liposomes in either the aqueous compartment (if it is water-soluble/hydrophilic) or within the lipid bilayer (if fat-soluble/lipophilic). Liposomes are available in several different forms, including conventional liposomes, cationic liposomes, fusogenic liposomes, ligand-targeted liposomes and long circulating (sterically stabilized, pegylated, or “stealth”) liposomes (SLs). Liposomes variously show different structures, dimensions, lipid composition and surface charge. They can be composed of several concentric bilayers separated by aqueous compartments, with an external lipid bilayer containing other ever smaller bilayers separated by water cavities, like an onion structure. In this case liposomes are called Multilamellar Vesicles (MLVs) and show a size range of 500 nm to 5 μm, or by only one phospholipid bilayer surrounding an aqueous compartment. In this case liposomes can be differentiated in small, large and giant vesicles depending upon their dimension: Small Unilamellar Vesicles (SUVs) if they have a 20 to 200 nm range size, Large Unilamellar Vesicles (LUVs) with a 200 to 1 μm range size and Giant Unilamellar Vesicles (GUVs) with a size larger than 1 μm. Finally, similar in dimension to MLVs there are multi-compartmental structures constituted by vesicles surrounded by other vesicles called Multi Vesicular Vesicles (MVVs).

Some of the primary lipids used to make liposomes are phospholipids and sphingolipids. Due to their amphiphilic nature, these molecules spontaneously self-assemble to form liposomes and other 3D structures when added to aqueous solutions. The shape or morphology of the 3D structures is dependent on a variety of different factors—for example, lipid composition, temperature, pH or the presence of other buffers, salts and sugars in the water.

Examples of phospholipids include glycerophospholipids such as phosphatidates, phosphatidylserine, phosphoinositides, phosphatidylinositol, phosphatidylinositol phosphate, phosphatidylinositol bisphosphate, phosphatidylinositol trisphosphate and phosphosphingolipids. In other examples the phospholipid lipid can be phosphatidylcholine (PC), phosphatidylethanolamine (PE), phosphatidylserine (PS) or phosphatidylglycerol (PG). Examples sphingolipids include ceramide phosphorylcholine, ceramide phosphorylethanolamine and ceramide phosphoryllipid. In other examples, the lipid is a glycolipid lipid. Examples of glycolipids include glyceroglycolipids, galactolipids, gangliosides, globosides, glycophosphosphingolipids and glycophosphatidylinositols.

In a preferred embodiment, the lipid-nucleic acid molecule complexes are cationic liposomes.

Cationic lipids typically feature a positively charged head group followed by hydrophobic tails of varying composition. Generally unsaturated, short (<30 monomer) hydrocarbon chains are associated with the highest transfection/transduction efficiencies. In aqueous environments, these lipids form liposomes with positively charged surfaces that complex with (but do not necessarily encapsulate) nucleic acid. These lipoplexes act to neutralize charge repulsion between the cell membrane and nucleic acid and promote endocytosis, similar to the mechanism of cationic polymers. Factors which can influence the efficiency of nucleic acid transfer include the ratio of lipid to nucleic acid, size of the lipoplex, and nature of the auxiliary lipid.

Cationic liposomes can be prepared by methods well known in the art or are available commercially, for example, Lipofectin, Lipofectace and Lipofectamine.

Lipofectamine consists of a 3:1 mixture of DOSPA (2,3-dioleoyloxy-N-[2(sperminecarboxamido)ethyl]-N,N-dimethyl-1-propaniminium trifluoroacetate) and DOPE (1,2-Dioleoyl-sn-glycero-3-phosphoethanolamine), which complexes with negatively charged nucleic acid molecules to allow them to overcome the electrostatic repulsion of the cell membrane. Lipofectamine's cationic lipid molecules are formulated with a neutral co-lipid (helper lipid). The DNA-containing liposomes (positively charged on their surface) can fuse with the negatively charged plasma membrane of living cells, due to the neutral co-lipid mediating fusion of the liposome with the cell membrane, allowing nucleic acid cargo molecules to cross into the cytoplasm for replication or expression.

Other lipids suitable for use in cationic liposomes and lipoplexes generally comprise mixtures of cationic lipids and neutral and zwitterionic lipids. These lipids include but are not limited to 1,2-dioleoylsn-glycero-3-phosphatidylcholine (DOPC), DiOleoylPhosphatidylEthanolamine (DOPE), 1,2-DiStearoyl-sn-glycero-3-PhosphoCholine (DSPC) PEI (polyethylenimine), N-[1-(2,3-dioleilooksy)-propyl]-N,N, N-trimethylammonium (DOTMA), methyl N-[1-(2,3-dioleilooksy)-propyl]-N,N,N-trimethylammonium chloride or 1,2-DiOleoyl-3-TrimethylAmmonium Propane (DOTAP), 2,3-dioleyloxy-N-[2(sperminecarboxamido)ethyl]-N,N-dimethyl-1-propanaminium trifluoroacetate (DOSPA), 3B—[N—(N′,N′-dimethylaminoethane)-carbamoyl] cholesterol hydrochloride (DC-cholesterol HCl) N,N-distearyl-N,N-dimethylammonium bromide (DDAB), N-(1,2-dimyristyloxyprop-3-yl)-N,N-dimethyl-N-hydroxyethyl ammonium bromide (DMRIE), N, N-dioleyl-N,N-dimethylammonium chloride (DODAC), diheptadecylamidoglycyl spermidine (DOGS), 1,3-dioleoyloxy-2-(6-carboxy-sperminyl)-propylamide (DOSPER), cholesterol, DC-cholesterol: 3B—[N—(N′,N′-dimethylaminoethane)-carbamoilo]-cholesterol, Arg-Chol: w-BOC-arginine glycinate cholesterol and mixtures thereof.

Liposomes and solid lipid nanoparticles (SLNs) (also known as lipid nanoparticles (LNPs)) are similar by design, but slightly different in composition and function. Both are lipid nanoformulations and excellent delivery vehicles, transporting cargo of interest within a protective, outer layer of lipids. In application, however, SLNs can take a variety of forms. An SLN is generally spherical in shape and consists of a solid lipid core stabilized by a surfactant. The core lipids can be fatty acids, acylglycerols, waxes, and mixtures of surfactants. Suitable lipids include triglycerides, diglycerides, monoglycerides, fatty acids, steroids, and waxes.

SLNs are liposome-like structures that can encapsulate a broad variety of nucleic acids (RNA and DNA); and as such, they are the most popular non-viral gene delivery system (for delivery of genetic payloads including siRNA and mRNA).

Traditional liposomes include one or more rings of lipid bilayer surrounding an aqueous pocket, but not all SLNs have a contiguous bilayer that would qualify them as lipid vesicles or liposomes. Some SLNs assume a micelle-like structure, encapsulating molecules in a non-aqueous core.

### Lipids

In addition to those mentioned above, other lipids suitable for use in the present invention include any lipids suitable for the preparation of lipid-based nanoparticles, particularly in the preparation of liposomes.

Lipids of the present disclosure are generally selected from the group comprising mono-, di-, or tri-glycerides, glycolipids, phospholipids, sphingolipids, ethanolamine and mixtures thereof. The lipids may also be pegylated. A lipid of the present disclosure can be made or obtained by any means known in the art including through both biological and synthetic means.

In some embodiments, the lipid has a chain length of C7-C35. In other examples the lipid is a long chain lipid, for example C13-C35 chain length. The lipid may be unsaturated or saturated. In other examples, the lipid chain length is C13 to C19. Examples of long chain lipids that can be used to prepare the lipid-nucleic acid molecule complex include mono-, di-, or tri-substituted glycerol, glycolipid, phospholipid, sphingolipid or ethanolamine derivatives of linear fatty acids.

Examples of linear fatty acids include: caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, ceroplastic acid and docosahexaenoic acid, pelargonic acid, undecylic acid, tridecylic acid, pentadecylic acid, margaric acid, nonadecylic acid, heneicosylic acid, tricosylic acid, pentacosylic acid, carboceric acid, nonacosylic acid, hentriacontylic acid, psyllic acid, tritriacontanoic acid, ceroplastic acid, heptatriacontylic acid, nonatriacontylic acid and tetracontylic acid. In some examples, the lipid is an unsaturated long chain lipid, for example, a long chain monoglyceride such as monoolein or monopalmitolein.

In other examples, the lipid is a branched lipid. Examples of branched lipids include fatty acid derivatives of mono-, di-, or tri-substituted glycerol, glycolipid, phospholipid or ethanolamine derivatives. Such fatty acids include mycolipanolic acid, mycoceranic acid, mycolipenic acid, micolipodienoic acid, mycocerosic acid, phthioceranic acids, dolichoic acids, phytanic acid, pristanic acid, from branched hydroxy fatty acids (mycolic acids), methoxymycolic acids, ketomycolic acids, 1-monomethyl branched fatty acids, 1-methyloctadec-12-enoic and 12-methyloctadec-10-enoic acids, cis-11-methyl-2-dodecenoic acid, tuberculostearic acid, phytomonic acid, 7-methyl-6-octadecenoic and 17-methyl-7-octadecenoic acids and laetiporic acid. Further examples of branched lipids relate to multi-branched lipids including isoprenoid-like lipids such as phytantriol and those derived from retinoic acid.

Typically, you need at least one charged lipid (e.g., ionizable, zwitterionic or cationic lipid) and at least one helper lipid to form a lipid-based nanoparticle, such as a LNP or liposome. One or more stabilization lipids may also be used.

The lipids may be used in any suitable molar ratio. Preferably when using an ionizable lipid, the lipids are used in the molar ratio of ionizable lipid and helper/neutral lipid:stabilisation lipid (e.g., cholesterol and or PEGylated lipid) of >50%:<50%. For example, the molar ratio of ionizable lipid:helper/neutral lipid:cholesterol:PEGylated lipid may be 46.3:9.4:42.7:1.6

In one example, the N:P ratio, i.e., the ratio of positively chargeable polymer amine (N=nitrogen) groups to negatively charged nucleic acid phosphate (P) groups is 6.

In one example, the following lipids may be used:
1. Ionizable Lipid—ALC-0315 (Acuitas Therapeutics). This may aid in the encapsulation of nucleic acids through electrostatic interactions.
2. Helper Lipid—DSPC 1,2-distearoyl-sn-glycero-3-phosphocholine. This may improve LNP stability and fusogenicity.
3. PEGylated lipid—ALC-0159 (Acuitas Therapeutics). This may prevent non-specific protein adsorption, particle aggregation and controls the resulting lipid nanoparticle (LNP) size.
4. Cholesterol. This may decrease the LNP permeability and increase its stability. This may also act as a cryoprotection additive when we freeze-dry the composition.

Other lipids may be used for the formation of lipid-based nanoparticles such as LNP (lipid nanoparticles or SLNs) or liposomes. Examples are given below.

### Charged Lipids

### (a) Cationic Lipids

Cationic lipids that may be used for lipid-based nanoparticle formulation include, for example, 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), Dimethyldioctadecylammonium bromide (DDAB), Commercial cationic agent Lipofectamine, which is composed of DOPE and 2,3-dioleyloxy-N-[2-(sperminecarboxamido)ethyl]-N,N-dimethyl-1-propanaminium trifluoroacetate (DOSPA), a cationic lipid containing quaternary ammonium and spermine, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N, N-bis(2-hydroxyethyl)-N-methylethan-1-aminium bromide (BHEM-Cholesterol).

### (b) Ionizable Lipids

Ionizable lipids that may be used for lipid-based nanoparticle formulation include, for example those that are protonated at low pH, which makes them positively charged, but that remain neutral at physiological pH, for example, (2S)-2,5-bis(3-aminopropylamino)-N-[2-(dioctadecylamino)acetyl]pentanamide (DOGS; Transfectam) N1-[2-((1S)-1-[(3-aminopropyl)amino]-4-[di(3-aminopropyl)amino]butylcarboxamido)ethyl]-3,4-di[oleyloxy]-benzamide (MVL5) DC-Cholesterol, N4-cholesteryl-spermine (GL67), 1,2-dilinoleyloxy-N,N-dimethyl-3-aminopropane (DLin-DMA), 2,2-dilinoleyl-4-dimethylaminoethyl-[1,3]-dioxolane (DLin-KC2-DMA), (6Z,9Z,28Z,31Z)-heptatriaconta-6,9,28,31-tetraen-19-yl 4-(dimethylamino) butanoate (DLin-MC3-DMA; MC3), di((Z)-non-2-en-1-yl) 9-((4-(dimethylamino)butanoyl)oxy)heptadecanedioate (L319), heptadecan-9-yl 8-((2-hydroxyethyl)(8-(nonyloxy)-8-oxooctyl)amino)octanoate (Lipid 5), heptadecan-9-yl 8-((2-hydroxyethyl)(6-oxo-6-(undecyloxy)hexyl)amino) octanoate (Lipid H (SM-102)) and ((4-hydroxybutyl)azanediyl)bis(hexane-6,1-diyl)bis(2-hexyldecanoate) (ALC-0315).

In addition to cationic or ionizable lipids, other lipid components, such as phospholipids, for example, phosphatidylcholine and phosphatidylethanolamine, polyethylene glycol (PEG)-functionalized lipids (PEG-lipids) for example, 1,2-dimyristoyl-rac-glycero-3-methoxypolyethylene glycol-2000 (PEG2000-DMG) and 1,2-distearoyl-rac-glycero-3-methoxypolyethylene glycol-2000 (PEG2000-DSG), 1,2-distearoyl-sn-glycero-3-phosphocholine (DSPC) can be used for lipid-based nanoparticle formulation.

### Cells

The compositions of the disclosure can be used to deliver nucleic acid molecules into cells for therapeutic, clinical and research application. The cells may be prokaryotic cells or eukaryotic cells. The cells may be from a single-celled organism or a multi-celled organism. In some cases, the cells are genetically engineered, e.g., the cells may be chimeric cells. The cells may be bacterial, fungi, plant, or animal cells, etc. The cells may be from a human or a non-human animal or mammal. For instance, if the cell is from an animal, the cell may be a cardiac cell, a fibroblast, a keratinocyte, a hepatocyte, a chondrocyte, a neural cell, an osteocyte, an osteoblast, a muscle cell, a blood cell, an endothelial cell, a stem cell, an immune cell (e.g., a T-cell, a B-cell, a macrophage, a neutrophil, a basophil, a mast cell, an eosinophil), etc. In some cases, the cell is a cancer cell. The cells may be non-adherent cell lines (e.g. L1.2, mouse immune B; Jurkat, human CD4+ T; Ramos, human immune B) or adherent cell lines (GPE86, mouse embryonic fibroblast), as well as primary cells. In a preferred embodiment, the cell is a primary cell such as a primary immune cell or stem cell. In one embodiment, the cell is a primary immune cell, for example, a T cell.

As used herein, the term “immune cells” refer to cells of the immune system, which defend the body against disease and foreign materials. Non-limiting examples of immune cells include dendritic cells, such as bone marrow-derived dendritic cells; lymphocytes, such as B cells, T cells, and natural killer cells; and macrophages. The immune cells may, in some embodiments, be derived from bone marrow, spleen, or blood from a suitable subject. For example, the immune cells may arise from a human or a non-human mammal, such as a monkey, ape, cow, sheep, goat, horse, donkey, llama, rabbit, pig, mouse, rat, guinea pig, hamster, dog, cat, etc. In a preferred embodiment, the immune cell is a T-cell, preferably a human T-cell.

As used herein, the term “stem cells” refers to clonogenic cells capable of both self-renewal and multilineage differentiation. Based on their origin, stem cells are categorised either as embryonic stem cells (ESCs) or as postnatal stem cells/somatic stem cells/adult stem cells (ASCs).

Embryonic stem cells (ESCs) can be derived from embryos that are 2-11 days old called blastocysts. They are totipotent—capable of differentiating into any type of cell including germ cells. ESCs are considered immortal as they can be propagated and maintained in an undifferentiated state indefinitely.

Adult stem cells (ASCs) are found in most adult tissues. They are multipotent—capable of differentiating into more than one cell type but not all cell types. Depending on their origin, AASCs can be further classified as hemopoetic stem cells (HSCs) and mesenchymal stem cells (MSCs). HSCs can be obtained either from cord blood or peripheral blood. MSCs are those that originate from the mesoderm layer of the fetus and in the adult reside in a variety of tissues such as the bone marrow stem cells (BMSCc), limbal stem cells, hepatic stem cells, dermal stem cells, etc. Stem cells have also been isolated from orofacial tissues which include adult tooth pulp tissue, pulp tissue of deciduous teeth, periodontal ligament, apical papilla, and buccal mucosa.

HSCs can be divided into a long-term subset, capable of indefinite self-renewal, and a short-term subset that self-renew for a defined interval. HSCs give rise to non-self-renewing oligolineage progenitors, which in turn give rise to progeny that are more restricted in their differentiation potential, and finally to functionally mature cells including myeloid (monocytes and macrophages, neutrophils, basophils, eosinophils, dendritic cells), erythroid (erythrocytes), megakaryocytic (platelets) and lymphoid lineages (T-cells, B-cells, NK-cells).

### Methods of Transfection/Transduction

The compositions provided herein may be used to transfect or transduce cells in vitro or ex vivo by any suitable means. In some embodiments, the lipid-nucleic acid molecule complex is released from the MOF shell by pH-induced targeted release prior to use in the transfection or transduction of cells. For example, the composition may be reconstituted in a physiologically acceptable liquid vehicle having a pH at which the MOF shell dissolves.

### Methods of Administration

The compositions provided herein may be administered to a subject including animals such as mammals or avians, or patient by any suitable means. As used herein, the term “patient” typically refers to a child or adult human in need of vaccination.

The composition may be reconstituted in a physiologically acceptable liquid vehicle to form an injectable solution or suspension, and then the injectable solution or suspension is injected into the subject or patient. The composition may be reconstituted directly in a hypodermic syringe or in a sterile vial or other container. The reconstituted composition may then be injected into the subject or patient, for example, by intramuscular, intradermal, or subcutaneous injection.

In some embodiments, the lipid-nucleic acid molecule complex is released from the MOF shell by pH-induced targeted release prior to administration to the subject or patient. For example, the composition may be reconstituted in a physiologically acceptable liquid vehicle having a pH at which the MOF shell dissolves.

### Examples

### Materials

All materials, zinc acetate dihydrate (≥99%), 2-methylimidazole (99%), sodium citrate dihydrate, citric acid, UltraPure™ DNase/RNase-Free Distilled Water, OptiMEM™ reduced serum media, (https://www.thermofisher.com/order/catalog/product/31985062), PureYield™ Plasmid Maxiprep System, were purchased commercially and used as is. All buffers were sterilized by autoclaving at 121ºC for 1 h. A Lipofectamine 3000 lipid transfection system were used for the pGFP DNA transfection. https://www.thermofisher.com/au/en/home/brands/product-brand/lipofectamine/lipofectamine-3000.html) A PureYield™ Plasmid Maxiprep System (promega.com.au) was used to prepare plasmid DNA, which is a pCAGGS-GFP plasmid of 5.5 kb size. Lipofectamine™ MessengerMAX™ mRNA Transfection Reagent was used as a control for the mRNA transfection experiments. (https://www.thermofisher.com/au/en/home/brands/product-brand/lipofectamine/lipofectamine-messengermax.html)

### Cell Culture

The technology/Transfection efficiency was tested in vitro in human cell line, Hela cells. Hela cells were maintained at 37° C., 5% CO₂ in complete cell culture medium containing MEM, 10% fetal calf serum (FCS) and 1% Pen/Strep (100 μg/mL penicillin and 100 units/mL streptomycin) and subcultured approximately every 4 d. One day prior to the transfection, cells were seeded at a density of 1.5×10⁵ cells per well in 24 well plates.

### Lipofectamine-DNA Complex Formation

An aliquot of pGFP (4000 ng/μl) was removed from −20° C. storage and diluted to a working solution of 500 ng/μl using UltraPure™ DNase/RNase-Free Distilled Water. To a 180 μL of OptiMEM™ media in an Eppendorf tube, 6.5 μl of this DNA was added and mixed using a vortex. To this, 6 μl of the p3000 component from the lipofectamine reagent system was added and mixed using a vortex. Finally, 7.5 μl of the lipofectamine 3000 reagent was added, the net 200 μl of solution was mixed and let to sit for 10-15 min before transfection or the next step of MOF formation on this complex.

### Preparation of Standard ZIF-8

A 100 μL of 2 methylimidazole (Hmlm) solution (64 mM prepared in OptiMEM™) was added to the Lipofectamine-DNA Complex formation prepared before and carefully mixed by pipetting. Next, a 100 μL solution of zinc acetate (ZnAc) dihydrate (16 mM) was quickly added and mixed using soft pipetting. Flocculates appeared immediately and the solution was left to sit over a period of 15 min at room temperature. The pellet was collected by centrifugation at 13,500 g for 5 min, the supernatant was collected for encapsulation efficiency assessment and the pellet was washed with 400 μl of OptiMEM™ media followed by collection of ZIF-8@Lipofectamine@DNA Complex using centrifuging as before. A higher precursor concentration, of 320 mM Hmlm and 80 mM zinc acetate and a lower precursor concentration, of 32 mM Hmlm and 8 mM zinc acetate were also assessed. The pellet was used as it is either for transfection experiment or stored in the required conditions for stability assessment. Throughout the study, the working volumes were maintained for comparison between the control and test samples, i.e. when a 200 μl of Lipofectamine@DNA solution in OptiMEM™ was used for the ZIF-8@Lipofectamine@DNA synthesis, the resulting pellet was reconstituted to make a 200 μl volume to perform the transfection study.

### Transfection Study

An in vitro transfection study was conducted to determine the degree of Lipofectamine@DNA complex integrity and functionality following the various ZIF-8@Lipofectamine@DNA composite preparations and storage conditions. While performing the 15 min incubations in previous steps, the media on cells plated in the 24 well plates was swapped with 450 μl of OptiMEM™ media in each well. The ZIF-8@Lipofectamine@DNA wet pellet was reconstituted using a 200 μl sodium citrate (pH 5.0, 50 mM) release buffer solution. The control Lipofectamine@DNA was used as it is. The transfection procedure required addition of 50 μL of either the Lipofectamine@DNA or the ZIF-8@Lipofectamine@DNA to the 24 well plate containing 1.5×10⁵ Hela cells in 450 μl of OptiMEM™ per well. After 5 h of incubation in a cell culture incubator at 37° C., 5% CO₂, the transfection media was removed and replaced with complete cell culture medium and the cells were left to translate and express GFP overnight in the cell culture incubator.

### Transfection Analysis

The transfected plate post-incubation for overnight (12 h) at 37° C., 5% CO₂ was moved into the BSC II and the culture media was carefully removed. The plate was rinsed with phosphate buffered saline (PBS) preheated to 37° C. It was then fixed with 4% paraformaldehyde (PFA) solution (300 μl each well) for 1 h at 37° C. PFA was removed, the plate was then washed twice with PBS before incubation with DAPI stain (1:2000) diluted in PBS for 10 min at room temperature. The plate was then washed twice with PBS water before reading at the microscope or storage in dark at 4° C. Cells were kept hydrated under a small volume of PBS and plates were read within 7 d of staining.

### Storage Stability Studies

Each of the wet pellet for the ZIF-8@Lipofectamine@DNA composite and the OptiMEM™ solution for the Lipofectamine@DNA complexes were placed at −80° C., −20° C., 4ºC, room temperature (regulated at approximately 25° C.) and 37° C. for a period of 1 weeks. At regular time intervals, transfection study was performed on three samples (n=3) from each formulation type stored at each of the three temperature conditions, respectively.

MessengerMax-EGFP mRNA Complex Formation.

An aliquot of EGFP mRNA was removed from its −40° C. storage and diluted to a working stock of 0.25 mg/mL using UltraPure™ DNase/RNase-Free Distilled Water. To a 5 μL of OptiMEM™ media in an Eppendorf tube, 0.15 μl of Lipofectamine™ MessengerMax™ was added and mixed using a vortex and left to incubate for 10 min at room temperature. In another Eppendorf tube, to a 4 μL OptiMEM media aliquot, 1 μL of mRNA (0.25 mg/mL) was added and incubated at room temperature for 5 min. The diluted mRNA and MessengerMax aliquots were mixed and incubated for 5 min at room temperature to get ˜10 μL MessengerMax-mRNA or Lipo@mRNA complex.

### Preparation of MOF@Lipo@mRNA

Four increasing precursor concentrations for ZIF-8, termed MOF-A, B, C, and D were synthesized and tested for their transfection efficiency and effect on cell viability, respectively.

Stock solutions of 26.26 mg/mL or 320 mM 2-methylimidazole (Hmlm) and 18.75 mg/mL or 80 mM, pH 7.4 Zinc acetate (ZnAc) dihydrate, pH 7.4 were prepared in OptiMEM media. MOF-A, B, C, D@Lipo@mRNA were prepared;
MOF A: to a 100 μL aliquot of Lipo@mRNA, 50 μL of 2 methylimidazole (Hmlm) solution in OptiMEM media(16 mM) was added and carefully mixed by pipetting. Next, a 50 UL solution of zinc acetate (ZnAc) dihydrate (4 mM) was quickly added and mixed using gentle pipetting.
MOF B: to a 100 μL aliquot of Lipo@mRNA, 50 μL of 2 methylimidazole (Hmlm) solution in OptiMEM media(32 mM) was added and carefully mixed by pipetting. Next, a 50 μL solution of zinc acetate (ZnAc) dihydrate (8 mM) was quickly added and mixed using gentle pipetting.
MOF C: to a 100 UL aliquot of Lipo@mRNA, 50 μL of 2 methylimidazole (Hmlm) solution in OptiMEM media(80 mM) was added and carefully mixed by pipetting. Next, a 50 μL solution of zinc acetate (ZnAc) dihydrate (20 mM) was quickly added and mixed using gentle pipetting.
MOF D: to a 100 UL aliquot of Lipo@mRNA, 50 μL of 2 methylimidazole (Hmlm) solution in OptiMEM media(160 mM) was added and carefully mixed by pipetting. Next, a 50 μL solution of zinc acetate (ZnAc) dihydrate (40 mM) was quickly added and mixed using gentle pipetting.

Flocculates appeared immediately and the solution was left to sit in the biological safety cabinet class II (BSC II) over a period of 15 min at room temperature. The resulting 200 μL MOF solutions were centrifuged at 13500×g for 5 min. The pellet was collected and washed with 200 μl OptiMEM. The centrifugation cycle was repeated. The pellet was either dispersed in 100 μl OptiMEM and stored or dispersed in 100 μl sodium citrate buffer (pH 5.0, 100 mM) to chelate the MOF and release the mRNA for use in the transfection experiment.

### Transfection Using MOF@Lipo@mRNA Complex

10 μl of the MOF@Lipo@mRNA sodium citrate solution was added to each well on a 96-cell culture plate (250 ng mRNA/100 μl/20,000 cells).

### LNP@mRNA (Lipoosomal Nanoparticle-mRNA) Preparation:

The LNP-mRNA complex was synthesized by mixing an aqueous solution of EGFP-mRNA with a lipid mixture dissolved in the organic phase (ethanol). The lipid LNP system consisted of 4 lipids, ALC-0315, ALC-0159, DSPC and, Cholesterol. Lipids were used in the molar ratio of: ionizable lipid (ALC-0315 (Acuitas Therapeutics)):helper/neutral lipid (DSPC 1,2-distearoyl-sn-glycero-3-phosphocholine):cholesterol:PEGylated lipid (ALC-0159 (Acuitas)) of 46.3:9.4:42.7:1.6. An aqueous solution of mRNA (ARCA modified EGFP mRNA; 996 nucleotides; 1 mg/ml in 1 mM Sodium Citrate, pH 6.4) (solution (a) and an Ethanolic (100%) solution of the 4 lipids (solution (b)) were prepared. Following preparation, the two solutions (a) and (b) were mixed in a 1:3 volumetric ratio (60 μl:180 μl).

Ethanolic/Organic Phase: A 60 μl ethanol solution containing the following mg of lipids (prepared from stock solutions of the lipids, prepared beforehand and stored at −40° C.-used within 3 months) was prepared: 0.43 mg ALC-0315, 0.05 mg ALC-0159, 0.09 mg DSPC and 0.2 mg Cholesterol.

Aqueous Phase: An aqueous phase at pH 4.0 was used. 12 mM Sodium Citrate Buffer at pH 4.0 was used.

To 150 μl of this buffer, 30 μl of mRNA stock (1 mg/ml) as supplied by the manufacturer (ApexBio; https://www.apexbt.com/arca-egfp-mrna.html) was added (1 mM Sodium Citrate, pH 6.4). A low pH buffer was used to ionize the lipid ALC-0315 and enable its electrostatic interaction with the mRNA. A N:P ratio of 6 was used. A N:P ratio of 2.0 was shown to be less efficient in transfection studies.

Citrate Buffer Prep (pH 4, 12 mM): For 100 mL, prepared 80 mL of RNAase free water in a suitable container. Added 119.1 mg of Sodium Citrate dihydrate to the solution. Then added 152.7 mg of Citric Acid to the solution. Adjusted solution to final pH 4.0 using HCl or NaOH. Added RNAase free water until volume reached 100 mL.

Synthesis: Ethanolic and Aqueous phase were mixed using pipetting. Two pipettes were used simultaneously to drop the solutions in an Eppendorf tube followed by mixing with a single pipette for at least 30× times. The obtained 240 μl solution (appears turbid) was then dialyzed against 1 litre of 100 mM Phosphate Buffered saline (PBS) at pH 7.4 to remove ethanol and replace the buffer solution. Dialysis was performed using Pur-A-Lyzer™ Mini Dialysis Kit, 10-250 μl, 6-8 kDa. Dialysis was performed for 1 h. Dialysis was done to remove ethanol and to raise the pH to 7.4 to assist with MOFencapsulation. Dialysis was used for the replacement of ethanol and the low pH dialysis buffer was used because MOF (esp. ZIF-8) formation requires rather neutral or slightly high pH conditions.

Post Dialysis, the 240 μl (60+180) of nanoparticle formulation was collected from the dialysis tube. This 240 μl of formulation contained 30 μg of mRNA; 125 ng/μl=mRNA concentration.

### Transfection Experiment (Control—Lipid@mRNA Complex)

Titration experiments have shown that there is no significant benefit in using the mRNA at more than 250 ng/well (20,000 cells/well/100 μl media) when performing transfection experiments in vitro.

The 240 μl of nanoparticle formulation was diluted 1:5 using OptiMEM media (low serum media) to give 25 ng/μl mRNA.

10 μl (250 ng mRNA) of this formulation was added to each well in a 96 well cell culture plate containing 20,000 cells in 90 μl media.

### TEM Analysis

The sample preparation for TEM analysis were performed as described previously. For TEM sample preparation, carbon-coated grids (EMSCF200H—CU-TH, ProSciTech) were glow discharged to render them hydrophilic. A 10 μl drop of each sample was applied to an upturned grid held in anti-capillary forceps, over moist filter paper, and left for 10 min to adsorb. The excess sample was then removed with filter paper. If stained, the grid was then inverted onto a drop of 2% uranyl acetate stain, on Parafilm, for 1 to 5 min. The grid was removed, the stain wicked away with filter paper and allowed to dry before viewing in the microscope. The samples were examined using a Tecnai 12 Transmission Electron Microscope (FEI, Eindhoven, The Netherlands) at an operating voltage of 120 kV. Images were recorded using a FEI Eagle 4k×4k CCD camera and AnalySIS v3.2 camera control software (Olympus.).

### Description of Results

In a typical experiment, for biomimetic mineralised growth of the MOF around the lipid-nucleic acid complex, MOF@lipo@DNA(pGFP) in this case, a frozen (−80° C.) vial of the nucleotide is completely thawed on ice. A liposomal complex is formed using OptiMEM™ media, following the manufacturers protocol. Any buffer, such as Tris, Hepes etc that can be used for efficient liposomal nanoparticle formation can be used at this stage. Then, an aqueous solution of the organic ligand is added to the lipo@pGFP followed by the addition of the metal salt solution (in OptiMEM™). In the presence of the lipo@pGFP complex, the reaction solution slowly begins to flocculate indicating MOF initiation which is left to form over a period of 15 min. The MOF@lipo@pGFP pellet is collected after centrifugation. The encapsulation efficiency is determined by measuring the cell transfection efficiency in both the supernatant and the pellet following release and comparing it with the control lipo@pGFP prepared alongside using a green fluorescent protein expression in Hela cells.

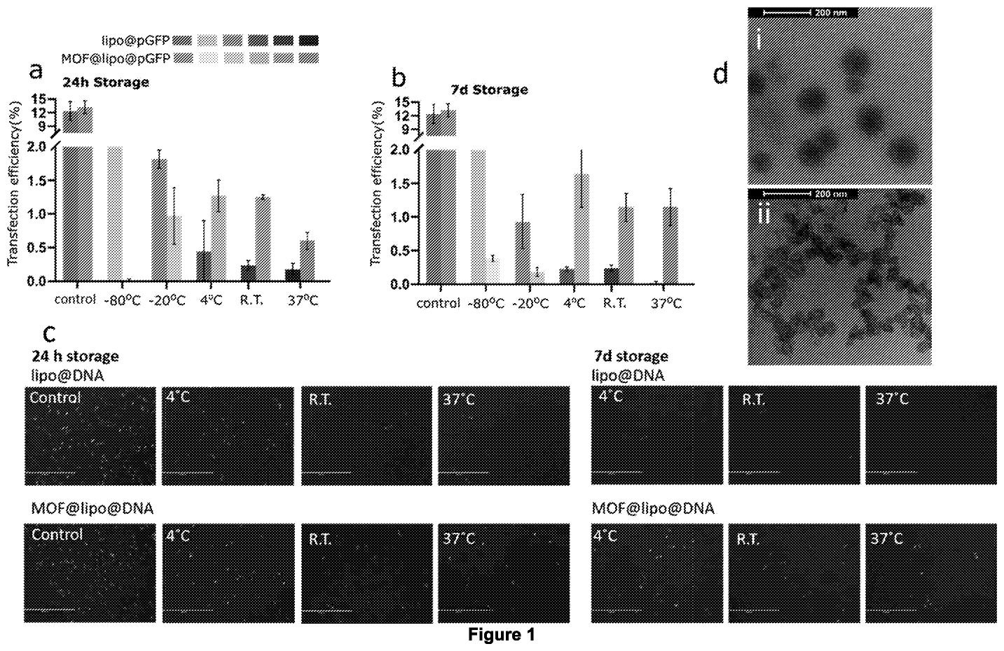

As shown in FIG. 1a, the MOF@lipo@pGFP complex retains complete transfection efficiency of the lipo@pGFP complex demonstrating 100% encapsulation. A 1.625 μg/mL of the pGFP DNA resulted in a mean 12.4% transfection of the total cells. The MOF@lipo@pGFP complex resulted in a mean 13.2% transfection. These complexes are then subjected to the storage conditions of −80° C., −20° C., 4° C., Room Temperature (R.T.) and 37° C. for 24 h and 7 d, respectively. The present inventors observed that at the freezing temperatures of −80° C. and −20° C. the MOF complex undergoes a freeze-stress leading to near total loss of the complex integrity. At 24 h, at 4ºC, R.T., and 37° C., the MOF@lipo@pGFP and the lipo@pGFP complexes demonstrate a mean 1.3% vs 0.4%, 1.3% vs 0.2% and 0.6 vs 0.2% transfection efficiency at 4° C., R.T. and 37° C., respectively. The MOF encapsulation provides the lipo@pGFP significant thermal protection.

At a relatively longer storage duration, the MOF mediated protection is even more significant with a mean 1.6% vs 0.2%, 1.1% vs 0.2% and 1.1% vs total loss of the MOF@lipo@pGFP and lipo@pGFP complex at 4° C., R.T. and 37° C., respectively.

The transmission electron microscopy (TEM) images in FIG. 1d show (i) individual <200 nm sized spherical particles, while in the MOF encapsulated form, (ii) MOF@lipo@pGFP appear as larger cluster of particles as small as 20 nm in size.

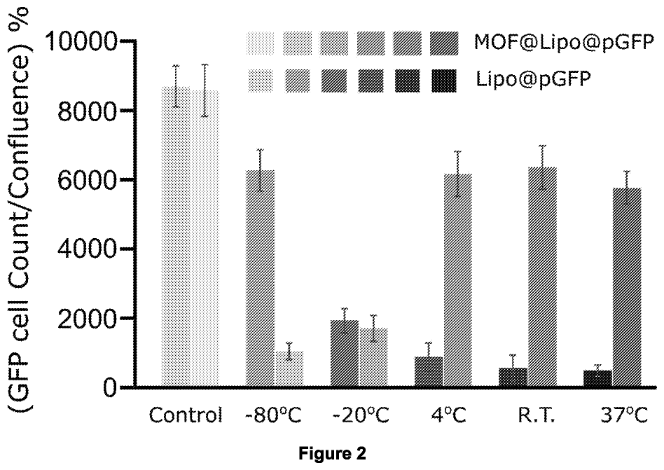

As shown in FIG. 2, a repeat 24 h storage experiment was performed with removal of the complexes from the cells after 5 hours (and addition of culture medium), resulting in improved transfection efficiency (cf FIG. 1 where the complexes were not removed prior to analysis). Transfection efficiency is expressed as the number of cell or cell counts of GFP expressing cells as a percent of total cell confluence. The results demonstrate repetitive, significant increase in thermal stability of MOF encapsulated liposomal complexes, MOF@lipo@pGFP compared with lipo@pGFP complexes without the MOF protection after the storage of the two complexes at −80° C., −20° C., 4° C., Room Temperature (R.T.) and 37° C. for 24 h against the efficiency of the freshly prepared complexes (control) at time t=0. The MOF@lipo@pGFP complex retains complete transfection efficiency of the lipo@pGFP complex demonstrating 100% encapsulation. In addition, the MOF complex after its 24 h storage at 4° C., Room Temperature (R.T.) and 37° C. still maintains the transfection efficiency similar to the lipo@pGFP complex stored at the −80° C. temperature.

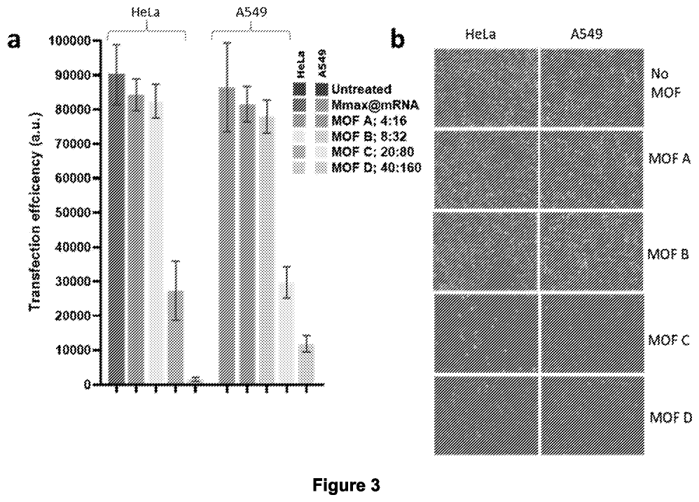

Proof-of-concept study demonstrating green fluorescent protein (EGFP) mRNA lipid complex encapsulated by a MOF with varying precursor concentrations (A-D, MOF precursor mM concentration ratios are included in the legend) and released prior to in-vitro transfection which was used as a surrogate efficacy assay. Lipofectamine MessengerMax (Mmax) was used as the transfection agent in this series of experiments. FIG. 3a shows analysis of Transfection efficiency (%) defined by the number of fluorescent cells per unit confluence in two cell lines, A549 and Hela. The results demonstrate the retention of structural and functional integrity of the lipid-mRNA complex encapsulated and released from MOF A and MOF B which were synthesized using lower precursor concentrations. The transfection efficiency for the Lipo@mRNA after release from MOF A and B is as efficient as the untreated Lipo@mRNA. The higher MOF precursor concentrations (MOF C and D) result is significantly lower transfection efficiency.

FIG. 3b shows representational images from cells treated with the No MOF (i.e. Lipo@mRNA; lipid-mRNA complex without MOF encapsulation) and MOF-A to D treated HeLa and A549 cells respectively. MOF A and MOF B show good transfection efficiency, indicating that the process of encapsulation and release does not impact on the transfection efficiency and the subsequent EGFP expression. The lower efficiency of MOF-C and MOF-D results from the lower cell viability resulting from higher precursor concentrations.

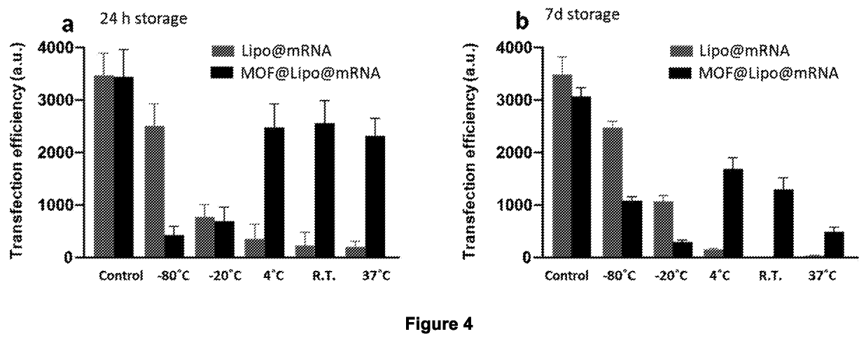

FIG. 4 shows the transfection efficiency of Lipo@mRNA and MOF-B@Lipo@mRNA samples after thermal challenges for 24 h and 7 days. MOF B had a ratio of 8 mM:32 mM metal:ligand concentration as previously described. The control samples confirm again that the process of MOF B encapsulation and release of the lipo@mRNA system maintains transfection efficiency. The thermal challenges demonstrate enhanced thermostability of the MOF B encapsulated lipid-mRNA composites after 24 h (FIG. 4a) and 7 days (FIG. 4b) of storage at 4° C., RT and 37° C. respectively compared with the unencapsulated, Lipo@mRNA composites. The present inventors have further performed this study with a more complex liposomal system.

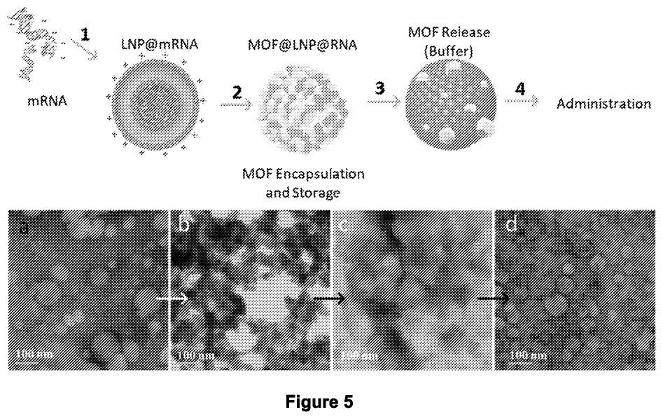

FIG. 5 shows proof-of-concept MOF encapsulated vaccine, a ZIF-8@Lipo@EGFP-mRNA formulation prepared using biomimetic mineralisation technique. The schematic illustrates the proposed mechanism. The present inventors first prepared a liposomal complex encapsulating a nucleic acid mRNA EGFP using 4 lipid components and an EGFP mRNA (step 1). The present inventors' postulate that the presence of these liposomal complexes in buffered solution increases the local concentration of MOF precursors, facilitating the formation of MOF prenucleation clusters, white. This leads to biomimetic growth of MOF crystals around the nucleic acid (step 2). Prior to administration, the MOF is released with addition of a buffer (Step 3) which leaves an intact LNP@mRNA ready for administration.

FIGS. 5(a-d) show transmission electron microscopy (TEM) visualization of encapsulation and release of the nucleic acid mRNA EGFP following those steps in the schematic. The liposomal complexes (a; imaged using uranyl acetate staining) are encapsulated using the ZIF-8 MOF forming an amorphous composite (b; unstained). A sodium citrate buffer (pH 5.0, 50 mM) was used to chelate the zinc ions causing MOF disintegration (c; unstained) releasing structurally intact liposomal complexes (d; uranyl acetate staining).

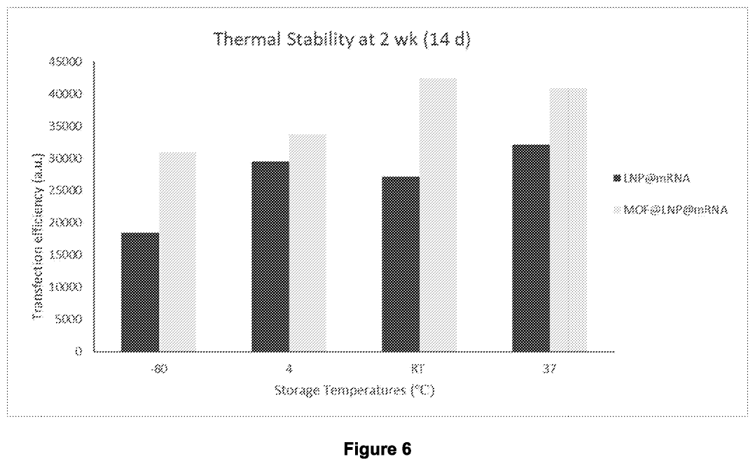

In FIG. 6, a single cationic lipid was used for complexation with the mRNA which was further MOF encapsulated and its storage stability analyzed. In FIG. 6, a more complex 4-lipid nanoparticle (LNP) system was demonstrated. The EGFP mRNA was used and the LNP as well as mRNA ratios were kept identical to the commercial formulation. The LNP@mRNA hence formed was then encapsulated using biomimetic mineralization technique to form MOF-A as described beforehand.

Both MOF A and LNP@mRNA formulations were stored for 14 days at −80° C., 4° C., RT and 37° C. respectively. The MOF encapsulation demonstrates significant enhancement in thermostability of the LNP@mRNA complexes.

A 4-lipid nanoparticle system was used. Briefly, the LNP@mRNA complex was synthesized by mixing an aqueous solution of EGFP-mRNA with a lipid mixture dissolved in the organic phase (ethanol). The lipid system consisted of 4 lipids, ALC-0315, ALC-0159, DSPC and, Cholesterol. The final LNP@mRNA formulation had an N/P ratio of 6 and the lipids were used to achieve a molar ratio of: ALC-0315 (ionizable lipid): ALC-0159 (neutral lipid):cholesterol:PEGylated lipid=46.3:9.4:42.7:1.6.

For MOF biomimetic mineralization, to a 100 μL aliquot of LNP@mRNA 50 μL of 2 methylimidazole (Hmlm) solution in OptiMEM media (32 mM) was added and carefully mixed by pipetting. Next, a 50 L solution of zinc acetate (ZnAc) dihydrate (8 mM) was quickly added and mixed using gentle pipetting. Flocculates appeared immediately and the solution was left to sit in the biological safety cabinet class II (BSC II) over a period of 15 min at room temperature. The pellet was collected by centrifugation at 13,500 g for 5 min. The pellet was washed with water followed by collection of ZIF-8@LNP@mRNA (MOF-A) using centrifugation as before. A higher precursor concentration of 64 mM Hmlm and 16 mM zinc acetate was also assessed (MOF-B).

Storage stability study was performed by storing aliquots of both the preparations at for 14 days at −80° C., 4ºC, RT and 37° C. respectively. Stability was determined by performing transfection study using the treated samples in A549 cells in vitro. MOF-A@LNP@mRNA had improved transfection efficiency compared to LNP@mRNA.

1. A stabilized molecular delivery composition comprising a lipid-nucleic acid molecule complex encapsulated within a Metal Organic Framework (MOF) shell.

2. The stabilized composition according to claim 1, wherein the nucleic acid is a DNA, RNA, oligonucleotide, antisense, CRISPR, siRNA, saRNA or microRNA molecule.

3. The stabilized composition according to claim 1 or claim 2, wherein the nucleic acid is a vector or a plasmid.

4. The stabilized composition according to any one of claims 1 to 3, wherein the lipid comprises a mixture of lipids.

5. The stabilized composition according to any one of claims 1 to 4, wherein the lipid comprises a cationic lipid.

6. The stabilized composition according to any one of claims 1 to 5, wherein the lipid comprises DOSPA (2,3-dioleoyloxy-N-[2-(sperminecarboxamido)ethyl]-N,N-dimethyl-1-propaniminium trifluoroacetate) and DOPE (1,2-Dioleoyl-sn-glycero-3-phosphoethanolamine).

7. The stabilized composition of any one of claims 1 to 6, wherein the composition is characterized as having improved stability over 4 weeks as compared to a comparative composition comprising the lipid-nucleic acid molecule complex without the MOF shell.

8. The stabilized composition of any one of claims 1 to 7, wherein the composition maintains at least 50% of its activity after 4 weeks of storage at temperatures up to 37° C.

9. The stabilized composition of any one of claims 1 to 8, wherein the composition maintains at least 75% of its activity after 4 weeks of storage at temperatures up to 37° C.

10. The stabilized composition of any one of claims 1 to 9, wherein the composition is characterized as having improved stability up to 12 weeks as compared to a comparative composition comprising the lipid-nucleic acid molecule complex without the MOF shell.

11. The stabilized composition of any one of claims 1 to 10, wherein the MOF is a zeolitic imidazolate framework (ZIF).

12. The stabilized composition of claim 11, wherein the ZIF is ZIF-8, ZIF-10, ZIF-90 or, ZIF-L.

13. The stabilized composition of claim 11 or claim 12, wherein the ZIF is ZIF-8.

14. The stabilized composition of any one of claims 1 to 10, wherein the MOF is aluminium fumarate.

15. The stabilized composition of any one of claims 1 to 14, wherein the composition is an amorphous composite.

16. The stabilized composition of any one of claims 1 to 15, wherein the composition comprises one or more excipients.

17. A method for producing a stabilized composition, the method comprising:
a. providing a lipid-nucleic acid molecule complex;
b. providing a ligand precursor;
c. providing a metal salt;
d. reacting the lipid-nucleic acid molecule complex, the ligand precursor and the metal salt to form a metal organic framework shell encapsulating the lipid-nucleic acid molecule complex.

18. The method of claim 17, wherein one or more of the lipid-nucleic acid molecule complex, the ligand precursor and the metal salt are provided in solution in one or mixed polar solvents.

19. The method of claim 18, wherein the solvent is water, alcohol, or other organic solvent, or buffer, or cell culture medium.

20. The method of claim 17 or claim 18, wherein the solution comprises one or more excipients.

21. The method of any one of claims 17 to 20, wherein the ligand precursor is 2-methylimidazole.

22. The method of any one of claims 17 to 21, wherein the ligand precursor is 80 to 640 mM 2-methylimidazole in buffer, or cell culture medium.

23. The method of any one of claims 17 to 22, wherein the metal salt is zinc acetate.

24. The method of any one of claims 17 to 23, wherein the metal salt is 20 to 160 mM zinc acetate dihydrate in buffer, or cell culture medium.

25. The method of any one of claims 17 to 24, wherein the metal salt: ligand precursor ratio is between 1:4 and 1:8.

26. The method of any one of claims 17 to 25, wherein the ligand precursor is fumaric acid.

27. The method of any one of claim 17 to 20, or 26, wherein the ligand precursor is 5 to 45 mM fumaric acid in buffer, or cell culture medium.

28. The method of any one of claims 17 to 20, or 26 and 27, wherein the metal salt is sodium aluminate.

29. The method of any one of claims 17 to 20, or 26 to 28, wherein the metal salt is 5 to 45 mM sodium aluminate in buffer, or cell culture medium.

30. The method of any one of claims 17 to 20 and 26 to 29, wherein the metal salt:ligand precursor ratio is 1:1.

31. The method of any one of claims 17 to 20, wherein the lipid-nucleic acid molecule complex, the ligand precursor and the metal salt solution are incubated for about 15 minutes.

32. The method of any one of claims 17 to 31, wherein the method further comprises centrifuging the reaction mixture of step (d) to pellet the metal organic framework encapsulating the lipid-nucleic acid molecule complex.

33. The method of any one of claims 17 to 32, wherein the method further comprises adding one or more excipients before the metal organic framework shell forms.

34. The method of claim 32 or claim 33, wherein the pellet is collected.

35. A method of preparation of the lipid-nucleic acid molecule complex composition of any one of claims 1 to 16 for administration or use, wherein the method comprises adding a release buffer to the composition to chelate the metal ions causing MOF disintegration, and thereby release the lipid-nucleic acid molecule complex.

36. The method of claim 35, wherein the release buffer is sodium citrate.
